SEQUENCE LISTING (16 sequences)

SEQ ID NO: 1 - DNA, 18 bp, artificial
  Fragile X gene promoter locus forward primer
ggggtcgaggggttgagt

SEQ ID NO: 2 - DNA, 18 bp, artificial
  Fragile X gene promoter locus reverse primer
accgcacgccccctaaca

SEQ ID NO: 3 - DNA, 18 bp, artificial
  Fragile X gene promoter locus reverse primer
accacacaccccctaaca

SEQ ID NO: 4 - DNA, 50 bp, artificial
  capture probe sequence for methylated converted amplified DNA, Fragile X gene promoter locus
cgcctccgtcaccgccgccgcccgcgctggccgtcgacccgccgcccgct

SEQ ID NO: 5 - DNA, 50 bp, artificial
  capture probe sequence for unmethylated converted amplified DNA, Fragile X gene promoter locus
cacctccatcaccaccaccacccacactcaccatcaacccaccacccact

SEQ ID NO: 6 - DNA, 37 bp, artificial
  capture probe sequence for methylated converted amplified DNA, Fragile X gene promoter locus
cgcctccgtcaccgccgccgcccgcgctcgccgtcga

SEQ ID NO: 7 - DNA, 40 bp, artificial
  capture probe sequence for unmethylated converted amplified DNA, Fragile X gene promoter locus
ttggttttatttttggtggagggttgtttttgagtgggtg

SEQ ID NO: 8 - DNA, 30 bp, artificial
  amelogenin specific forward primer
gcggccaccttactcatattatacttaaca

SEQ ID NO: 9 - DNA, 30 bp, artificial
  amelogenin specific reverse primer
gcggcgttgatggttggttttaagtttgtg

SEQ ID NO: 10 - DNA, 40 bp, artificial
  capture probe sequence for AMEX specific bisulphite converted amplified DNA
gtttttgttgaaatattagtgattaagtggtataggagag

SEQ ID NO: 11 - DNA, 50 bp, artificial
  capture probe sequence for the AMEY specific bisulphite converted amplified DNA
ggtaaaatgtaaagattaggtaaaattattaattttgggtaaataatttt

SEQ ID NO: 12 - DNA, 22 bp, artificial
  SNRPN specific forward primer
tgtttatggtttttagaggttt

SEQ ID NO: 13 - DNA, 22 bp, artificial
  SNRPN specific reverse primer
ccaccaacacaactaaccttac

SEQ ID NO: 14 - DNA, 22 bp, artificial
  SNRPN specific reverse primer
ccaccgacacaactaaccttac

SEQ ID NO: 15 - DNA, 37 bp, artificial
  capture probe sequence for unmethylated SNRPN specific bisulphite converted amplified DNA
tatgtttgtgtggttgtagaggtaggttggtgtgtat

SEQ ID NO: 16 - DNA, 44 bp, artificial
  capture probe sequence for methylated SNRPN specific bisulphite converted amplified DNA
ccgacgaccgctccactctacgccaaactcgctacaacaacgac

(19) United States
(12) Patent Application Publication
(10) Pub. No.: US 2011/0046009 A1
(43) Pub. Date: Feb. 24, 2011

(54) METHODS FOR DETECTING DNA METHYLATION USING ENCODED PARTICLES

(75) Inventor: JAMES K. MOORE, STOW, MA (US)

Correspondence Address: GIFFORD, KRASS, SPRINKLE, ANDERSON;& CITKOWSKI, P.C., P.O. BOX 7021, TROY, MI, 48007-7021, US

(73) Assignee: PERKINELMER HEALTH SCIENCES, INC., WALTHAM, MA (US)

(21) Appl. No.: 12/862,491

(22) Filed: Aug. 24, 2010

Related U.S. Application Data
(60) Provisional application No. 61/236,473, filed on Aug. 24, 2009.
(60) Provisional application No. 61/285,557, filed on Dec. 11, 2009.

(51) Int. Cl. C40B 30/04; C12Q 1/68; C40B 40/06; C07H 21/04

(52) U.S. Cl. 506/9; 435/6; 506/16; 536/24.31

(57) ABSTRACT

Methods for detecting the methylation status of a target genomic locus are provided. Methods described allow for simultaneous assay of multiple cytosines in a target genomic locus. Assays of multiple cytosines in a target genomic locus provide detection of an aggregate cytosine methylation state of the target genomic locus. Methods to detect methylation of a genomic locus associated with a disease or disorder characterized by aberrant methylation of the genomic locus, such as, but not limited to, Fragile X mental retardation syndrome, Prader-Willi syndrome, Angelman sydrome, Beckwith-Wiedemann syndrome, and Russell-Silverman syndrome, diabetes, cancer, multiple sclerosis or schizophrenia are described herein.

### REFERENCE TO RELATED APPLICATIONS

This application claims priority to U.S. Provisional Patent Application Ser. Nos. 61,236,473, filed Aug. 24, 2009 and 61/285,557, filed Dec. 11, 2009, the entire content of both of which is incorporated herein by reference.

### FIELD OF THE INVENTION

Methods described generally relate to assays for determining the methylation status of a target nucleic acid. In specific aspects, described methods relate to assays for determining whether a target genomic sequence is methylated within a plurality of specified nucleotides. In further specific aspects, described methods relate to assays for determining whether a particular target genomic sequence in a DNA sample is aberrantly methylated within a plurality of specified nucleotides compared with unaffected DNA.

### BACKGROUND OF THE INVENTION

Epigentic variation in methylation has been used to differentiate numerous biological processes, diseases and disorders. Several genetic disorders are characterized by abnormal methylation of DNA in the promoter region of the associated gene. These disorders are associated with developmental problems including cognitive deficiencies, neurological and metabolic deficiencies. Various cancers also are characterized by abnormal methylation of DNA, and methylation differences have been used to detect the presence of cancerous cells. There is a need for medical tests for determining methylation status of DNA, for example, in prenatal testing to characterize fetal health conditions, in post-natal testing when unexplained developmental problems are observed in the infant, and in carrier testing. In prenatal testing, methylation status can be relevant when examining fetal cells for suspected disorders and aneuploidies. One approach for identifying fetal cells in maternal blood is based on methylation differences between fetal (for example, placental derived) and maternal samples. It would be useful to test for some or all of these disorders simultaneously in a multiplexed assay format.

Examples of disorders characterized by methylation of DNA include Fragile X syndrome, Prader-Willi syndrome, Angelman sydrome, Beckwith-Wiedemann syndrome, and Russell-Silverman syndrome. In the case of Fragile X syndrome, the methylation of the gene promoter is accompanied by an expansion and methylation of a trinucleotide repeat region adjacent to the gene. Common methods for testing for Fragile X syndrome include evaluating of the extent of the trinucleotide repeat region using Southern blotting and evaluating the methylation state of this region using restriction enzyme analysis. Such methods are labor- and time-intensive. Other methods include methylation-specific PCR and methylation-specific MLPA (multiplex ligation-dependent probe amplification). In these methods, in which quantitative or real-time PCR is used to combine an amplification and detection step, only four reporters can be used simultaneously. This limits the disorder panels that can be addressed to only two disorders when it is desired to test affected and unaffected outcomes (i.e., methylated and un-methlylated states).

Other assays have been developed for detecting methylation of single bases in multiplexed assay formats. An example of this type of assay is the Golden Gate Methylation assay (Illumina, San Diego Calif.), which uses two probes to interrogate each single-base methylation site in the target. The sequences of these probes are exactly complementary to the bisulfite-converted target sequence adjacent to the interrogation site in order for the hybridization—extension—ligation steps to work. This is straightforward to design for isolated CpG sites, but challenging in CpG-rich regions such as the Fragile X promoter site where a CpG site occurs on average every 7 bases, and the sequence of the bisulfite-converted probe sites can be variable. Assuming that all CpG sites will be methylated at once simplifies the design, but the literature shows that while the majority of CpG sites are methylated when the promoter is methylated overall, individual CpG sites remain unmethylated in a pattern that varies from sample to sample. Thus, assays of this type can be non-applicable to assessing the aggregate methylation state of longer CpG-containing DNA segments, such as promoter regions.

### SUMMARY OF THE INVENTION

Methods for detecting an aggregate cytosine methylation state in a target genomic locus are provided which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine; labeling the amplified converted DNA with a label; contacting the labeled amplified converted DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate; detecting the label; and detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.

The encoded substrates are encoded particles according to embodiments described herein.

The encoded substrates are elements of a microarray according to embodiments described herein.

Amplifying converted DNA using primers that flank the target genomic locus to produce amplified converted DNA is by polymerase chain reaction according to embodiments described herein.

Methods provided herein including amplifying converted DNA using primers that flank the target genomic locus to produce amplified converted DNA can include use of at least one composite primer; at least one non-methylation specific primer and/or at least one methylation-specific primer.

Labeling the amplified converted DNA with a label can be performed during the amplifying or after the amplifying. Labeling the amplified converted DNA with a label during the amplifying can include using at least one labeled primer and/or labeled nucleotides. Labeling the amplified converted DNA with a label after the amplifying can include binding of a labeled probe specific for the amplified converted DNA.

According to methods described herein, the target genomic locus assayed to determine methylation state is associated with a disorder selected from the group consisting of: Fragile X mental retardation syndrome, Prader-Willi syndrome, Angelman sydrome, Beckwith-Wiedemann syndrome, Russell-Silverman syndrome, diabetes, cancer, multiple sclerosis or schizophrenia.

The first capture probe selective for the unprotected amplification product and associated with a first encoded substrate is an oligonucleotide capture probe according to embodiments. The second capture probe selective for the unprotected amplification product and associated with a second encoded substrate is an oligonucleotide capture probe according to embodiments.

Methods are provided according to embodiments described herein which further include treating a reference DNA sample comprising the target genomic locus with bisulfite to convert unmethylated cytosine to uracil, producing converted reference DNA; amplifying converted reference DNA using primers that flank the target genomic locus to produce amplified converted reference DNA, wherein an unprotected amplification product is produced when the target genomic locus sample lacks methylated cytosine, and a protected amplification product is produced when the target genomic locus contains methylated cytosine; labeling the amplified converted reference DNA with a label; contacting the labeled amplified converted reference DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate; detecting the label; detecting the encoding of the first and second encoded substrates, wherein association of the detected label with the first encoded substrate indicates that the target genomic locus in the reference DNA sample has a negative aggregate cytosine methylation state and wherein association of the detected label with the second encoded substrate indicates that the target genomic locus in the reference DNA sample has a positive aggregate cytosine methylation state; and comparing the aggregate cyosine methylation states of the DNA sample and the reference DNA sample.

Embodiments described herein further include amplifying converted DNA using primers that flank a second target genomic locus to produce amplified converted DNA, wherein an unprotected amplification product is produced when the second target genomic locus in the DNA sample lacks methylated cytosine, and a protected amplification product is produced when the second target genomic locus in the DNA sample contains methylated cytosine; labeling the amplified converted DNA with a label; contacting the labeled amplified converted DNA with (i) a third capture probe, wherein the third capture probe is selective for the unprotected amplification product of the second target genomic locus and is associated with a third encoded substrate, and (ii) a fourth capture probe, wherein the fourth capture probe is selective for the protected amplification product of the second target genomic locus and is associated with a fourth encoded substrate;detecting the label; and detecting the encoding of the third and fourth encoded substrates, wherein association of the detected label with the third encoded substrate indicates that the second target genomic locus in the DNA sample has a negative aggregate cytosine methylation state and wherein association of the detected label with the fourth encoded substrate indicates that the second target genomic locus in the DNA sample has a positive aggregate cytosine methylation state.

Embodiments optionally further include amplifying converted DNA using primers that flank a gender-specific target genomic locus to produce a gender-specific amplified converted DNA; contacting the gender-specific amplified converted DNA with (i) a first capture probe selective for the female-specific amplification product, the first capture probe attached to a first gender-specific encoded substrate, and (ii) a second capture probe selective for the male-specific amplification product, the second capture probe attached to a second gender-specific encoded substrate; labeling the gender-specific amplified converted DNA with a label; detecting the label; and detecting the encoding of the encoded substrates to produce a gender result, wherein association of the detected label with the first gender-specific encoded substrate indicates that the DNA sample was obtained from a female subject and wherein association of the detected label with the second gender-specific encoded substrate indicates that the DNA sample was obtained from a male subject.

The gender-specific target genomic locus is an amelogenin genomic locus according to embodiments of methods provided herein.

Methods for detecting an aggregate cytosine methylation state in a target genomic locus, are provided according to embodiments described herein which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine; labeling the amplified converted DNA with a label; contacting the labeled amplified converted DNA with (i) a first capture probe, wherein the first capture probe is selective for the unprotected amplification product to produce a complex of the first capture probe and the unprotected amplification product and (ii) a second capture probe, wherein the second capture probe is selective for the protected amplification product to produce a complex of the second capture probe and the protected amplification product; associating the complex of the first capture probe and the unprotected amplification product and with a first encoded substrate; associating the complex of the second capture probe and the protected amplification product and with a second encoded substrate; detecting the label; and detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.

Kits for detecting the aggregate methylation state of a target genomic locus are provided herein which include an encoded particle set comprising attached capture probes specific for a target genomic locus containing one or more cytosines to be assayed for methylation state; and instructional material for use of the encoded particle set in a method of detecting cytosine methylation state according to embodiments of the present invention. Kits for detecting the aggregate methylation state of a target genomic locus are provided herein which include an encoded particle set comprising attached capture probes specific for a target genomic locus containing two or more cytosines to be assayed for methylation state; and instructional material for use of the encoded particle set in a method of detecting aggregate cytosine methylation state according to embodiments of the present invention.

Compositions are provided according to embodiments described herein including a complex of a capture probe attached to an encoded substrate and labeled unprotected amplification product, wherein the complex indicates that the unprotected amplification product is derived from a target genomic locus in a DNA sample having a negative aggregate cysteine methylation state.

Compositions are provided according to embodiments described herein including a complex of a capture probe attached to an encoded substrate and labeled protected amplification product, wherein the complex indicates that the protected amplification product is derived from a target genomic locus in a DNA sample having a positive aggregate cysteine methylation state.

Methods for screening a subject for a disease or disorder characterized by methylation of a genomic locus are provided according to embodiments of the present invention which include treating a DNA sample obtained from the subject comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine; labeling the amplified converted DNA with a label; contacting the labeled amplified converted DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate; detecting the label; and detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.

Methods for detecting methylated cytosine in a target genomic locus are provided according to embodiments described herein which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus in the DNA sample lacks methylated cytosine, and a protected amplification product is produced when the target genomic locus in the DNA sample contains methylated cytosine; labeling the amplified converted DNA with a label; contacting the labeled amplified converted DNA with (i) a first capture probe selective for the unprotected amplification product, the first capture probe attached to a methylation-negative encoded substrate, and (ii) a second capture probe selective for the protected amplification product, the second capture probe attached to a methylation-positive encoded substrate, thereby forming a complex of (i) the first capture probe attached to the methylation-negative encoded substrate and the labeled unprotected amplification product or (ii) the second capture probe attached to the methylation-positive encoded substrate and the labeled protected amplification product; detecting the label; and detecting the encoding of the methylation-negative and methylation-positive encoded substrates to produce a first result, wherein association of the detected label with the methylation-negative encoded substrate indicates that the target genomic locus in the DNA sample lacks methylated cytosine and wherein association of the detected label with the methylation-positive encoded substrate indicates that the target genomic locus in the DNA sample includes methylated cytosine.

According to embodiments of provided methods, the DNA sample is a human DNA sample.

According to embodiments of provided methods, the DNA sample is obtained from a human subject suspected of having or at risk of having a disorder characterized by aberrant methylation of a genomic locus, such as, but not limited to, Fragile X mental retardation syndrome, Prader-Willi syndrome, Angelman sydrome, Beckwith-Wiedemann syndrome, and Russell-Silverman syndrome, diabetes, cancer, multiple sclerosis or schizophrenia.

According to embodiments of provided methods, the DNA sample is obtained from a human subject suspected of having or at risk of having Fragile X mental retardation syndrome. According to embodiments of provided methods, the DNA sample is obtained from a human subject suspected of having or at risk of having Prader-Willi syndrome. According to embodiments of provided methods, the DNA sample is obtained from a human subject suspected of having or at risk of having Angelman sydrome. According to embodiments of provided methods, the DNA sample is obtained from a human subject suspected of having or at risk of having Beckwith-Wiedemann syndrome. According to embodiments of provided methods, the DNA sample is obtained from a human subject suspected of having or at risk of having Russell-Silverman syndrome. According to embodiments of provided methods, the DNA sample is obtained from a human subject suspected of having or at risk of having cancer. According to embodiments of provided methods, the DNA sample is obtained from a human subject suspected of having or at risk of having diabetes. According to embodiments of provided methods, the DNA sample is obtained from a human subject suspected of having or at risk of having multiple sclerosis. According to embodiments of provided methods, the DNA sample is obtained from a human subject suspected of having or at risk of having schizophrenia.

Methods for detecting methylated cytosine in a target genomic locus are provided according to embodiments described herein which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus in the DNA sample lacks methylated cytosine, and a protected amplification product is produced when the target genomic locus in the DNA sample contains methylated cytosine; labeling the amplified converted DNA with a label; contacting the labeled amplified converted DNA with (i) a first capture probe selective for the unprotected amplification product, the first capture probe attached to a methylation-negative encoded substrate, and (ii) a second capture probe selective for the protected amplification product, the second capture probe attached to a methylation-positive encoded substrate, thereby forming a complex of (i) the first capture probe attached to the methylation-negative encoded substrate and the labeled unprotected amplification product or (ii) the second capture probe attached to the methylation-positive encoded substrate and the labeled protected amplification product; detecting the label; detecting the encoding of the methylation-negative and methylation-positive encoded substrates to produce a first result, wherein association of the detected label with the methylation-negative encoded substrate indicates that the target genomic locus in the DNA sample lacks methylated cytosine and wherein association of the detected label with the methylation-positive encoded substrate indicates that the target genomic locus in the DNA sample includes methylated cytosine; treating a reference DNA sample comprising the target genomic locus with bisulfite to convert unmethylated cytosine to uracil, producing converted reference DNA; amplifying converted reference DNA using primers that flank the target genomic locus to produce amplified converted reference DNA, wherein an unprotected amplification product is produced when the target genomic locus in the reference DNA sample lacks methylated cytosine, and a protected amplification product is produced when the target genomic locus in the reference DNA sample contains methylated cytosine; labeling the amplified converted reference DNA with a label; contacting labeled amplified converted reference DNA with (i) the first capture probe selective for the unprotected amplification product, the first capture probe attached to a methylation-negative encoded substrate, and (ii) the second capture probe selective for the protected amplification product, the second capture probe attached to a methylation-positive encoded substrate, thereby forming a complex of (i) the first capture probe attached to the methylation-negative encoded substrate and the labeled unprotected amplification product or (ii) the second capture probe attached to the methylation-positive encoded substrate and the labeled protected amplification product; detecting the label; detecting the encoding of the methylation-negative and methylation-positive encoded substrates to produce a reference result, wherein association of the detected label with the methylation-negative encoded substrate indicates that the target genomic locus in the reference DNA sample lacks methylated cytosine and wherein association of the detected label with the methylation-positive encoded substrate indicates that the target genomic locus in the reference DNA sample includes methylated cytosine; and comparing the first result and the reference result to detect differences in methylation of the DNA sample compared to the reference DNA sample.

Methods for detecting methylated cytosine in a target genomic locus are provided according to embodiments described herein which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus in the DNA sample lacks methylated cytosine, and a protected amplification product is produced when the target genomic locus in the DNA sample contains methylated cytosine; labeling the amplified converted DNA with a label; contacting the labeled amplified converted DNA with (i) a first capture probe selective for the unprotected amplification product, the first capture probe attached to a methylation-negative encoded substrate, and (ii) a second capture probe selective for the protected amplification product, the second capture probe attached to a methylation-positive encoded substrate, thereby forming a complex of (i) the first capture probe attached to the methylation-negative encoded substrate and the labeled unprotected amplification product or (H) the second capture probe attached to the methylation-positive encoded substrate and the labeled protected amplification product; detecting the label; detecting the encoding of the methylation-negative and methylation-positive encoded substrates to produce a first result, wherein association of the detected label with the methylation-negative encoded substrate indicates that the target genomic locus in the DNA sample lacks methylated cytosine and wherein association of the detected label with the methylation-positive encoded substrate indicates that the target genomic locus in the DNA sample includes methylated cytosine; amplifying converted DNA using primers that flank a second target genomic locus to produce a second amplified converted DNA, wherein a second unprotected amplification product is produced when the second target genomic locus in the DNA sample lacks methylated cytosine, and a second protected amplification product is produced when the second target genomic locus in the DNA sample contains methylated cytosine; labeling the second amplified converted reference DNA with a label; contacting the second labeled amplified converted DNA with (i) a first capture probe selective for the second unprotected amplification product, the first capture probe attached to a methylation-negative encoded substrate, and (ii) a second capture probe selective for the second protected amplification product, the second capture probe attached to a methylation-positive encoded substrate, thereby forming a complex of (i) the first capture probe attached to the methylation-negative encoded substrate and the second labeled unprotected amplification product or (ii) the second capture probe attached to the methylation-positive encoded substrate and the second labeled protected amplification product; detecting the label; and detecting the encoding of the methylation-negative and methylation-positive encoded substrates to produce a second result, wherein association of the detected label with the methylation-negative encoded substrate indicates that the second target genomic locus in the DNA sample lacks methylated cytosine and wherein association of the detected label with the methylation-positive encoded substrate indicates that the second target genomic locus in the DNA sample includes methylated cytosine.

According to embodiments described herein, the methylation-positive encoded substrate including the second capture probe selective for the second unprotected amplification product includes encoding distinguishable from the methylation-positive encoded substrate including the first capture probe selective for the first unprotected amplification product.

Methods for detecting methylated cytosine in a target genomic locus are provided according to embodiments described herein which include treating a DNA sample comprising the target genomic locus with bisulfite to convert unmethylated cytosine to uracil, producing converted DNA; amplifying converted DNA using primers that flank the target genomic locus to produce amplified converted DNA, wherein an unprotected amplification product is produced when the target genomic locus in the DNA sample lacks methylated cytosine, and a protected amplification product is produced when the target genomic locus in the DNA sample contains methylated cytosine; labeling the amplified converted DNA with a label; contacting the labeled amplified converted DNA with (i) a first capture probe selective for the unprotected amplification product, the first capture probe attached to a methylation-negative encoded substrate, and (ii) a second capture probe selective for the protected amplification product, the second capture probe attached to a methylation-positive encoded substrate, thereby forming a complex of (i) the first capture probe attached to the methylation-negative encoded substrate and the labeled unprotected amplification product or (ii) the second capture probe attached to the methylation-positive encoded substrate and the labeled protected amplification product; detecting the label; detecting the encoding of the methylation-negative and methylation-positive encoded substrates to produce a first result, wherein association of the detected label with the methylation-negative encoded substrate indicates that the target genomic locus in the DNA sample lacks methylated cytosine and wherein association of the detected label with the methylation-positive encoded substrate indicates that the target genomic locus in the DNA sample includes methylated cytosine;amplifying converted DNA using primers that flank a gender-specific target genomic locus to produce a gender-specific amplified converted DNA; contacting the gender-specific amplified converted DNA with (i) a first capture probe selective for the female-specific amplification product, the first capture probe attached to a female encoded substrate, and (ii) a second capture probe selective for the male-specific amplification product, the second capture probe attached to a male encoded substrate, thereby forming a complex of: (i) the first capture probe attached to the female encoded substrate and the female-specific amplification product or (ii) the second capture probe attached to the male encoded substrate and the male-specific amplification product; labeling the gender-specific amplified converted DNA with a label; detecting the label; and detecting the encoding of the female and male encoded substrates to produce a gender result, wherein association of the detected label with the female encoded substrate indicates that the DNA sample was obtained from a female subject and wherein association of the detected label with the male encoded substrate indicates that the DNA sample was obtained from a male subject.

### BRIEF DESCRIPTION OF THE DRAWINGS

FIG. 1 is a schematic process flow chart of an exemplary assay as described herein;

FIG. 2 is a schematic drawing showing the PCR design of one exemplary assay as described herein;

FIG. 3 is a schematic drawing showing a single strand of the DNA product of PCR of one exemplary assay as described herein;

FIG. 4 is a schematic drawing showing a single oligonucleotide capture probe immobilized on an encoded particle according to an exemplary assay as described herein, and a single strand PCR product molecule from the previous steps specifically bound to the capture probe;

FIG. 5 is a schematic process flow chart of an exemplary process for the preparation of one pair of the encoded particles (or combined bead set) with immobilized probes for protected and unprotected converted PCR products of a target genomic locus according to an exemplary assay as described herein;

FIG. 6 is a schematic process flow chart of an exemplary process for the preparation of a multiplex bead set for assaying the methylation status of a plurality of n target genomic loci according to an exemplary assay as described herein;

FIG. 7 is a schematic process flow chart showing an exemplary hybridization assay portion of the process described in FIG. 1 with more detail;

FIG. 8a is a graph showing an example of data generated by an exemplary assay, showing the assay fluorescent signal from the methylation-positive encoded particles increasing with increasing inputs of PCR product made from a methylated sample, while the signal from the methylation-negative encoded particles remains low at all input levels; the graph shows the response with a linear DNA input axis;

FIG. 8b is a graph showing an example of data generated by an exemplary assay described herein, showing the assay fluorescent signal from the methylation-positive encoded particles increasing with increasing inputs of PCR product made from a methylated sample, while the signal from the methylation-negative encoded particles remains low at all input levels; the graph shows the same data as in FIG. 8a, here with a logarithmic DNA input axis;

FIG. 9a is a graph showing an example of data generated by an exemplary assay described herein, showing the assay fluorescent signal from the methylation-negative encoded particles increasing with increasing inputs of PCR product made from an unmethylated sample, while the signal from the methylation-positive encoded particles remains low at all input levels; the graph shows the response with a linear DNA input axis;

FIG. 9b is a graph showing an example of data generated by an exemplary assay described herein, showing the assay fluorescent signal from the methylation-negative encoded particles increasing with increasing inputs of PCR product made from an unmethylated sample, while the signal from the methylation-positive encoded particles remains low at all input levels; the graph shows the same data as in FIG. 9a, here with a logarithmic DNA input axis;

FIG. 10 is a schematic drawing showing overview of an embodiment of the described methods for determining the methylation state of a target genomic region;

FIG. 11A is a schematic drawing showing an overview of a pair of “simple” primers useful in various embodiments of the described methods for determining the methylation state of a target genomic region;

FIG. 11B is a schematic drawing showing an overview of a pair of “composite” primers useful in various embodiments of the described methods for determining the methylation state of a target genomic region;

FIG. 12 is a graph showing an example of data generated by an exemplary assay described herein, showing the ability to distinguish between Fragile X-affected and unaffected male subjects;

FIG. 13 is a graph showing an example of data generated by an exemplary assay described herein, showing the ability to distinguish between Fragile X-affected and unaffected female subjects;

FIG. 14 is a schematic drawing of the relevant gene regions of AMEX and AMEY, showing the common sequence used to anneal the PCR primers and the variable sequence used to identify the X and Y chromosomes;

FIG. 15 is a schematic process flow chart of an exemplary process for multiplex detection of methylation at the FMR promoter and gender identification;

FIG. 16 is a graph showing an example of data generated by an exemplary multiplex assay described herein, showing the ability to distinguish between Fragile X-affected and unaffected subjects;

FIG. 17 is a graph showing an example of data generated by an exemplary multiplex assay described herein, showing the ability to distinguish the gender of subjects; and

FIG. 18 is a graph showing an example of data generated by an exemplary assay described herein, showing the ability to distinguish between unaffected subjects and subjects affected with the Prader-Willi or Angelman syndromes.

The drawings are not to scale and the relative dimensions of various elements in the drawings are depicted schematically and not to scale.

### DETAILED DESCRIPTION OF THE INVENTION

Described herein are methods for detecting the methylation status of a target genomic locus.

The term “target genomic locus” refers to the region or segment of a genomic DNA molecule assayed to detect methylation of the region or segment of a genomic DNA molecule. In the methods described herein, the target genomic locus is amplified using an amplification technique, such as PCR, after treatment of genomic DNA with bisulfite. Accordingly, the target genomic locus is the segment of genomic DNA that is amplified using the selected primers, and can contain a promoter element or portion thereof.

Described herein are methods for determining the methylation state of a target genomic locus that contains one or more CpG islands. CpG islands are genomic DNA features that are well-known in the art. Simply stated, CpG islands are genomic regions that contain multiple CpG sites, which are sites having a cytosine nucleotide next to a guanine nucleotide in the linear sequence of bases along a length of DNA. In mammalian genomes, CpG islands are typically 300-3,000 base pairs in length, and often occur at or near the transcription start of genes. In humans, approximately 1% of DNA bases undergo methylation and about 10-80% of CpG dinucleotides are methylated. Methylation of CpG islands occurs during normal regulation within mammalian cells, and aberrant methylation can occur in, and therefore be indicative of, diseases such as Fragile X mental retardation syndrome, Prader-Willi syndrome, Angelman sydrome, Beckwith-Wiedemann syndrome, and Russell-Silverman syndrome. Other diseases associated with aberrant methylation include diabetes, cancer, multiple sclerosis and schizophrenia.

According to methods described herein a sample is assayed to detect aberrant genomic methylation that occurs in, and is indicative of, a diseases or disorder characterized by aberrant methylation, such as, but not limited to, Fragile X mental retardation syndrome, Prader-Willi syndrome, Angelman sydrome, Beckwith-Wiedemann syndrome, and Russell-Silverman syndrome diabetes, cancer, multiple sclerosis and schizophrenia. Target genomic loci aberrantly methylated in these and other diseases and disorders are well-known in the art. Examples of specific genomic loci known to be aberrantly methylated in affected individuals are described in Genc et al., Nucleic Acids Research, 28:2141-2152, 2000; Zeschnigk et al., Human Mol. Genetics, 6:387-395, 1997; and Esteller et al., Cancer Research, 60:4366-4371, 2000.

In the methods described herein, the target genomic locus is typically a region of DNA near, often upstream, or within a gene associated with a particular disorder or disease, which contains at least one CpG island that contains methylated cytosines in an individual who manifests the disorder or disease. For example, several disorders or disease conditions are characterized by at least one CpG island containing methylated cytosines in an individual having the disorder or disease and unmethylated in an individual who is unaffected by the disorder or disease. Since methylation patterns can vary somewhat from cell to cell in a sample from a single individual, assay of a single cytosine to determine if it is methylated can return an ambiguous result. Methods according to the present invention allow for simultaneous assay of multiple cytosines in a target genomic locus, in contrast to previously published assays which are capable of assaying only a single cytosine. Assays of multiple cytosines in a target genomic locus therefore provides detection of an “aggregate cytosine methylation state” of the target genomic locus. The term “aggregate cytosine methylation state” refers to the status of two or more cytosines in a target genomic locus as methylated or unmethylated. Thus, as is used herein, the term “aggregate” means that two or more cytosine residues are contained within a region of nucleic acid that is assessed by the methods.

Methods according to the present invention are not limited to assay of an “aggregate cytosine methylation state” of a target genomic locus containing two or more cytosines to be assayed for methylation status. Methods are provided for assay of the methylation state of a single cytosine according to embodiments described herein.

The target genomic locus can be in any form, such as chromosomal DNA, mitochondrial DNA, cDNA, microdissected chromosomal DNA, an insert in a vector illustratively including a bacterial artificial chromosome, yeast artificial chromosome, human artificial chromosome, cosmid, plasmid, phagemid, phage DNA and fosmid.

The term “target genomic locus” as used herein encompasses DNA molecules in any form including single-stranded, double-stranded, oligonucleotide or polynucleotide.

The target genomic locus is typically contained within a genomic DNA sample, which can be obtained from an individual, such as from a bodily sample, for example, blood, such as whole blood and dried blood spots; buccal swab; skin tissue; urine; saliva; tissue; and the like, and cell lines derived therefrom. A prenatal genomic DNA sample can be obtained from amniotic fluid, products of conception, blastocysts and blastomeres, corionic villi, placental cells, fetal cells and fetal DNA circulating in maternal blood. Archived samples extracted from formalin-fixed, paraffin-embedded (FFPE) pathology samples are also sources of sample genomic DNA that can be used in the methods described herein. Sample and/or reference genomic DNA can also be obtained from in vitro sources such as cell lines.

Thus, the genomic DNA sample can be obtained from any suitable source. Particular methods described herein involve using a genomic DNA sample from an individual subject. The genomic DNA sample can be obtained from any source, including, but not limited to, a human, a non-human mammal, a vertebrate, an invertebrate, a microorganism, or a plant. The genomic DNA sample can be obtained from one or more cells ex vivo or in vitro. For example, the genomic DNA sample can be obtained from cultured cells, including, but not limited to, cell lines, primary cells or laboratory manipulated cells such as recombinant cells.

Methods described herein include typically include a genomic DNA sample which is a “test” sample, that is, a genomic DNA sample containing a target genomic locus having an unknown methylation status. According to embodiments of methods described herein a genomic DNA sample which is a “reference” sample, that is, a genomic DNA sample containing a target genomic locus having a known methylation status, is included.

Methods of obtaining genomic DNA from these or other sources are well known in the art. Genomic DNA samples are obtained by methods known in the art, for instance, as described in J. Sambrook and D. W. Russell, Molecular Cloning: A Laboratory Manual, Cold Spring Harbor Laboratory Press; 3rd Ed., 2001 or F. M. Ausubel, Ed., Short Protocols in Molecular Biology, Current Protocols; 5th Ed., 2002. Genomic DNA samples may also be obtained commercially and/or using commercial kits for isolation of genomic DNA.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; and Herdewijn, P. (Ed.), Oligonucleotide Synthesis: Methods and Applications, Methods in Molecular Biology, Humana Press, 2004.

The methods for detecting methylation described herein involve treating the DNA sample containing the target genomic locus with bisulfite to convert unmethylated cytosine to uracil. Treatment of DNA with bisulfite to convert unmethylated cytosine to uracil is well-known in the art, see for example Clark et al., Nucl. Acids Res., 22(15):2990-2997, 1994. In procedures for treatment of DNA with bisulfite to convert unmethylated cytosine to uracil, DNA is typically denatured and treated with sodium bisulfite, causing unmethylated cytosines to convert to uracils while methylated cytosines remain unchanged. According to embodiments of methods of determining the methylation status of a target genomic locus, the genomic DNA sample including the target genomic locus is treated with bisulfite to convert unmethylated cytosine to uracil, producing “bisulfite-treated” DNA, also called “converted DNA”. Amplification of the converted DNA is performed to produce “amplified converted DNA.” When the bisulfite-treated DNA is amplified, such as by PCR, the uracils are amplified as thymidines, while the methylated cytosines are amplified as cytosines. Thus, based on the sequence of amplified converted DNA, such as PCR amplified converted DNA, made using bisulfite-treated DNA as template, the methylation state of the target genomic locus can be determined.

The “bisulfite” used to convert unmethylated cytosine to uracil is typically sodium bisulfite, although other sulfites can be used, such as, but not limited to, potassium bisulfite, magnesium bisulfite and ammonium bisulfite.

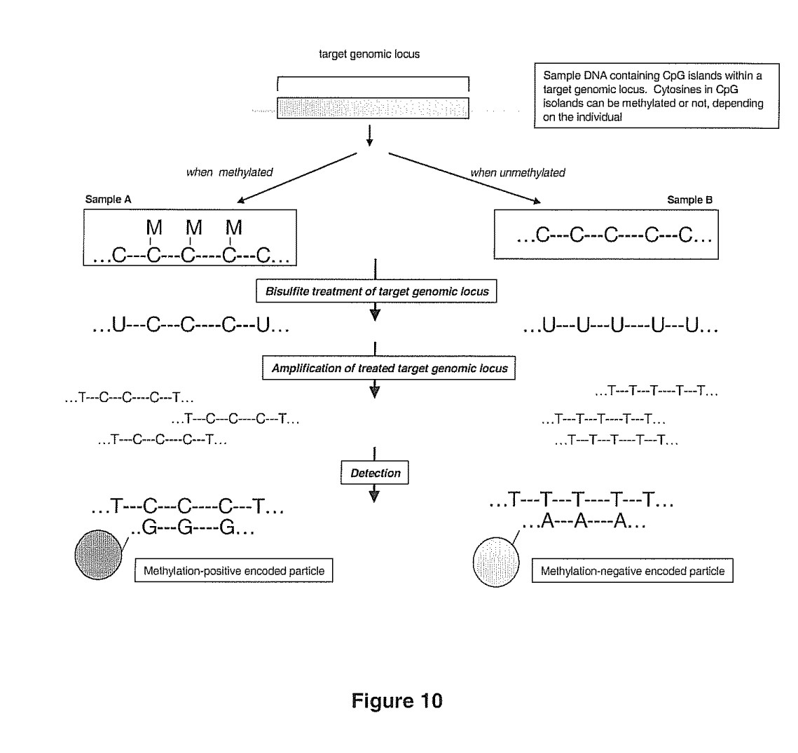

Referring to FIG. 10, a target genomic locus is depicted to contain a DNA sequence containing CpG islands having different methylation states in Samples A (methylated) and B (unmethylated). Upon bisulfite treatment, the methylated cytosines (shown as M-C) in Sample A are shown to be “protected” and unmethylated cytosines (C) are “unprotected” and converted to uracils (U), while in Sample B the unmethylated cytosines are “unprotected” and converted to uracils.

Referring again to FIG. 10, uracils contained in bisulfite-treated DNA from Samples A and B, are amplified as thymidines during PCR amplification. Amplification products, such as PCR products, produced using bisulfite-treated DNA as template can convey information about the methylation state of the original DNA samples. The term “amplified converted DNA” refers to the product of a process of copying a converted DNA template.

The term “unprotected amplification product” is used herein to describe amplified converted DNA in which the target genomic locus lacked methylation, while the term “protected amplification product” is used to describe amplified converted DNA in which the target genomic locus contained methylation. The term “unprotected PCR product” is used herein to describe PCR amplified converted DNA in which the target genomic locus lacked methylation, while the term “protected PCR product” is used to describe PCR amplified converted DNA in which the target genomic locus contained methylation.

An encoded substrate is provided which includes attached capture probes specific for amplified converted DNA wherein the target genomic locus in the genomic DNA sample was methylated. Another encoded substrate is provided which includes attached capture probes specific for amplified converted DNA wherein the target genomic locus in the genomic DNA sample was unmethylated. The amplified converted DNA and the capture probes are brought into contact under binding conditions and the amplified converted DNA binds to one of the substrates, depending on the methylation status of the target genomic locus in the genomic DNA sample, via specific binding to the capture probes.

The amplified converted DNA is labeled and the label is used to detect binding of the amplified converted DNA with capture probes which are attached to the encoded substrate. Detection of binding of the amplified converted DNA with capture probes and detection of the encoding of the substrates allows for determination of the methylation status of the target genomic locus in the genomic DNA sample.

Provided herein is a method for detecting methylation within a target genomic locus containing one or more CpG islands. The method involves treating a DNA sample containing the target genomic locus with bisulfite to convert unmethylated cytosine to uracil; amplifying a segment of the bisulfite-treated DNA using primers that flank the target genomic locus, wherein an unprotected PCR product is produced when the target genomic locus lacked methylation, and a protected PCR product is produced when the target genomic locus contained methylation; contacting resultant amplified segments with (i) an oligonucleotide probe selective for the unprotected PCR product, the probe being attached to a methylation-negative encoded particle, and (ii) an oligonucleotide probe selective for the protected PCR product, the probe being attached to a methylation-positive encoded particle, such that a complex of PCR product hybridized to a respective oligonucleotide probe attached to an encoded particle can produce a signal, detecting signals associated with the methylation-negative and methylation-positive encoded particles, thereby detecting the presence or absence of methylation within the target genomic locus.

Methods for detecting the methylation status of a target genomic locus are useful, for example, when determining whether the target genomic locus is aberrantly methylated, correlating with a disease or disease carrier genotype and/or phenotype. Methylation status also can be used to distinguish fetal cells from maternal cells in mixed samples, such as maternal blood.

The presently described methods employ detectable labels that generate signals correlating with the methylation status of a target genomic locus, and thus allow determination of the methylation status of a target genomic locus. Thus, the methods described herein can be used for understanding the methylation state of genomic regions associated with health conditions in individuals.

The term “aberrant methylation” a target genomic locus refers to methylation which differs from normal methylation of the target genomic locus, particularly aberrant methylation of the target genomic locus associated with a disease or disorder. Aberrant methylation of a target genomic locus can be detected using a normally methylated reference target genomic locus in embodiments of methods described herein. Aberrant methylation of a target genomic locus can be detected by comparison of methylation status detected using methods described herein with known methylation status of the normal target genomic locus. The term “normal” refers to the predominate methylation status of the target genomic locus found in unaffected subjects.

Some differences in methylation between individuals are a reflection of age, developmental stage and the like. In such case, methylation differences are not considered to be aberrant and can be detected using the methods described herein. In such cases, methylation status of a target genomic locus can be compared to a reference using methods described herein to detect differences or similarities of methylation status indicative of age, developmental stage and the like.

Methods for detecting the methylation status of a target genomic locus are useful in assays to determine the effect of a test agent on methylation of the target genomic locus.

The methods described herein can be formatted to yield a methylation-positive or methylation-negative result; an extent of methylation; a comparative extent of methylation, and the like; and combinations thereof. As is described in the Examples below, the methods can be used to assess the relative amounts of both the unmethylated and the methylated forms of genomic DNA in the promoter region of a disorder-linked gene.

Amplification

According to embodiments of methods of determining the methylation status of a target genomic locus, amplification of the converted DNA is performed to produce amplified converted DNA.

Amplification of a target genomic locus is achieved using an in vitro amplification method. The term “amplification method” refers to a method for copying a template nucleic acid, thereby producing nucleic acids which include copies of all or a portion of the template target nucleic acid.

Amplification methods included in embodiments of the present invention are those which include template directed primer extension catalyzed by a nucleic acid polymerase using a pair of primers which flank the target genomic locus, illustratively including, but not limited to, Polymerase Chain Reaction (PCR), reverse-transcription PCR (RT-PCR). ligation-mediated PCR (LM-PCR), phi-29 PCR, and other nucleic acid amplification methods, for instance, as described in C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Appropriate reactions conditions for synthesis of a primer extension product include presence of suitable reaction components including, but not limited to, a polymerase and deoxynucleoside triphosphates. Such reaction conditions and components are well-known in the art, for example as described in V. Demidov et al., DNA Amplification: Current Technologies and Applications, Taylor & Francis, 2004.

Primers

Methods described herein involve amplifying a segment of the bisulfite-treated DNA using primers that flank the target genomic locus. The term “primer” refers to a nucleic acid that is capable of acting as a site of initiation of synthesis of a primer extension product under appropriate reaction conditions. Selection of primers used in amplification methods will depend on the nucleotide sequence of regions that flank the target genomic locus. An oligonucleotide primer is typically about 10-30 contiguous nucleotides in length, although shorter or longer oligonucleotides are sometimes useful. An oligonucleotide primer is completely or substantially complementary to a region of a template nucleic acid such that, under hybridization conditions, the oligonucleotide primer anneals to the complementary region of the template nucleic acid. Design of oligonucleotide primers suitable for use in amplification reactions is well known in the art, for instance as described in A. Yuryev et al., PCR Primer Design, Humana Press, 2007.

Primer design for amplification of a target nucleic acid is well-known to those of skill in the art. Primers for amplification of a target nucleic acid are designed according to well-known methods and criteria. For instance, the annealing temperature of the primers should be about the same, within a few degrees, the primers should not form dimers with each other and the primers should not form secondary structures, such as hairpins. Methods and considerations for primer design and amplification procedures are described in detail in Yuryev, A., PCR Primer Design, Methods in Molecular Biology, vol. 42, Human Press, 2007; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Amplified converted DNA optionally contains additional materials such as, but not limited to, nucleic acid sequences, functional groups for chemical reaction and detectable labels, present in the primers and not present in the original converted DNA template. Such primer-derived materials add functionality such as a detectable label, primer binding sites for additional amplification reactions and/or a functional group for chemical bonding to a substrate.

In embodiments of the present invention a pair of primers used in amplification includes primers which flank a target genomic locus, that is, one of the primers has a nucleotide sequence complementary to a region of the target genomic locus upstream of one or more CpG islands and a second primer of the primer pair has a nucleotide sequence complementary to a region of the target nucleic acid downstream of the one or more CpG islands.

According to embodiments of the present invention, at least one of the primers used in amplification is a “methylation specific primer.” The term “methylation specific primer” refers to a primer which specifically hybridizes to a region of the target genomic locus including one or more methylated cytosines and does not specifically hybridize to the same site when the cytosine or cytosines are unmethylated.

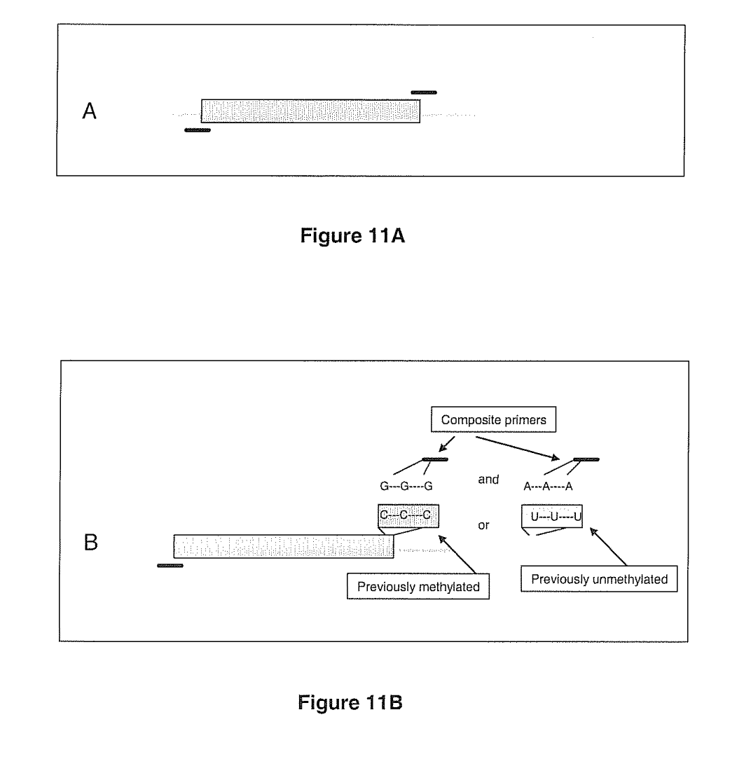

Referring to FIG. 11, the primer pair can be simple or composite, depending on the CpG content of the flanking sequences of the target. A simple pair is used when it is possible to design two primers that flank the target genomic locus and can amplify bisulfite-converted and non-converted sequences (FIG. 11A). A composite pair can be designed, for example, to compensate for low-density methylation in the flanking sequences. Specifically, one primer of the composite pair can be designed to be complementary to a bisufite-treated unmethylated sequence, while the second can be designed to be complementary to the bisufite-treated methylated sequence. These primers are mixed together to form a composite primer. FIG. 11B depicts an embodiment in which a simple primer is used on the 5′ flanking region of the target genomic locus while a composite primer is used on the 3′ flanking region. In this example the 3′ (reverse) primer is a composite of two primers, one with guanines in positions complementary to cytosines in the bisulfite-treated target genomic sequence having methylation. The other primer has adenines in those positions so as to be complementary to the uracils produced from bisulfite-treatment of target genomic sequence lacking methylation. These two primers are mixed together as a composite primer capable of priming the PCR reaction from either methylated or unmethylated DNA samples. Alternative embodiments include using composite primers on both the 5′ and 3′ regions of the target genomic locus and using a composite primer on the 5′ region and simple primer on the 3′ region.

Another option is a primer pair that is degenerate at the loci of nucleotides that vary with the methylation state.

The 5′ (forward) primer in FIG. 11B is a simple, single primer that primes the PCR reaction regardless of the methylation state of the target genomic locus.

Capture Probes

Capture probes specific for the amplified converted DNA are associated with a solid or semi-solid substrate for capture of the amplified converted DNA to the substrate. Capture probes can be in any form which allows for attachment of the capture probes to the substrate and specific capture of the amplified converted DNA.

According to some embodiments, capture probes are nucleic acids which include a nucleic acid sequence complementary to the amplified converted DNA, wherein the capture probes are specific for the “protected” amplified converted DNA, or for the “unprotected” amplified converted DNA.

Capture probes attached to a substrate can be single-stranded and/or double-stranded nucleic acids. In particular embodiments, where double-stranded nucleic acids capture probes are bound to the substrate, they are denatured and rendered single stranded after immobilization to the substrate for preparation for use in certain embodiments of assay methods. Optionally, double stranded nucleic acid probes are denatured prior to immobilization and the single stranded nucleic acids are then bound to the substrate.

In particular embodiments, the capture probes are oligonucleotides complementary to the amplified converted DNA, wherein the oligonucleotide capture probes specifically hybridize to the “protected” amplified converted DNA, or the “unprotected” amplified converted DNA.

As will be appreciated by one of skill in the art, hybridization conditions are used which allow for specific hybridization of a capture probe to the “protected” amplified converted DNA, or the “unprotected” amplified converted DNA, but not both. Further, hybridization conditions can be designed using well-known methods in the art to accommodate a degree of variability in methylation state of a target genomic locus between cells in a sample allowing for specific hybridization of a capture probe to the “protected” amplified converted DNA, or the “unprotected” amplified converted DNA, but not both.

Methods described herein involve contacting amplified converted DNA, such as PCR amplified converted DNA, with oligonucleotide capture probes that are selective for methylation states. The oligonucleotide capture probes are designed to hybridize with the unprotected PCR product (indicative of the target genomic locus lacking methylation) and/or the protected PCR product (indicative of the target genomic locus containing methylation).

Other types of capture probes that can be used include locked nucleic acids (LNAs), peptide nucleic acids (PNAs), DNA fragments and other DNA binding partners that specifically hybridize or otherwise specifically bind to DNA.

In some embodiments the substrates used are encoded particles and each of the different capture probes is attached to an encoded particle having a detectable signature that uniquely identifies the oligonucleotide. Capture probes that bind to unprotected PCR product are attached to encoded particles referenced herein as “methylation-negative encoded particles”, and probes that bind to protected PCR product are attached to encoded particles referenced herein as “methylation-positive encoded particles.” Thus, when a DNA sample contains a target genomic locus that is methylated, the resulting amplified PCR product will be protected, and can bind to a methylation-positive encoded particle via a unique probe specific for one or more methylated CpG dinucleotides within the target genomic region. Conversely, when a DNA sample contains a target genomic locus that is unmethylated, the resulting amplified PCR product will be unprotected, and can bind to a methylation-negative encoded particle via a unique probe specific for one or more unmethylated CpG dinucleotides within the target genomic locus (see FIG. 10 for a pictoral representation, not to scale).

A method of assaying sample genomic DNA is provided which includes two or more encoded particle sets encoded such that each particle of each encoded particle set is detectably distinguishable from each particle of each other encoded particle set. The encoded particles of a first particle set include attached capture probes which specifically capture “protected” amplified converted DNA corresponding to methylated target genomic locus. The encoded particles of a second particle set include attached capture probes which specifically capture “unprotected” amplified converted DNA corresponding to unmethylated target genomic locus.

Methods including two or more particle sets can be used in multiplex or separate assay formats.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100%, or completely, complementary to a region of the nucleotide sequence 5′-TTAGCTGG-3′. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize. The terms “hybridization” and “hybridized” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution.

The terms “specific hybridization” and “specifically hybridizes” refer to hybridization of a particular nucleic acid to a complementary nucleic acid without substantial hybridization to nucleic acids other than the complementary nucleic acid in a sample.

Detection of Binding of Amplified Converted DNA to Capture Probes

The amplified converted DNA is labeled with a label independent of the methylation status of the target genomic locus. The label is also termed a “target-detection label” herein and is used to detect binding of the amplified converted DNA with capture probes which are attached to encoded particles.

In particular embodiments, the label, that is, the “target-detection label,” is incorporated in at least one primer used in amplifying the target genomic locus to produce the amplified converted DNA.

In some embodiments, the “target-detection label” is present in nucleotides used in amplifying the target genomic locus to produce the amplified converted DNA.

In further embodiments, the amplified converted DNA is labeled with a “target-detection label” directly or indirectly by binding of a probe which specifically hybridizes to the amplified converted DNA. The term “probe” refers to an oligonucleotide that specifically hybridizes to amplified converted DNA of interest. The term “probe” refers to single-stranded and double-stranded oligonucleotides. A probe typically includes a label such that specific hybridization of the probe with the amplified converted DNA labels the amplified converted DNA.

Thus, amplified converted DNA can be labeled with a “target-detection label” during amplification or after amplification.

In the methods described herein, a complex of a PCR amplified converted DNA hybridized to a respective oligonucleotide capture probe attached to an encoded particle can produce a signal. The signal is indicative of binding between the oligonucleotide probe and the PCR amplified converted DNA, and can be, for example, a signal generated by a detectably labeled material associated with the oligonucleotide capture probe.

Another exemplary scenario is detecting a signal generated by a detectably labeled material associated with the PCR amplified converted DNA. A specific example described below is binding of streptavidin-phycoerythrin reporter to one or more biotin moieties incorporated into the PCR amplified converted DNA. In this specific example, detection of a phycoerythrin fluorescence signal associated with a methylation-negative encoded particle is indicative of an unmethylated state of the target genomic locus, whereas detection of a phycoerythrin fluorescence signal associated with a methylation-positive encoded particle is indicative of a methylated state of the target genomic locus.

Labels

The term “label” refers to a substance that can be measured and/or observed, visually or by any appropriate direct or indirect method illustratively including, but not limited to, spectroscopic, optical, photochemical, biochemical, enzymatic, electrical and/or immunochemical methods of detection, to indicate presence of the label. Non-limiting examples of labels that can be used in conjunction with methods described herein illustratively include a fluorescent moiety, a chemiluminescent moiety, a bioluminescent moiety, a magnetic particle, an enzyme, a substrate, a radioisotope and a chromophore.

For example, nucleotides, nucleotide analogs and/or primers can be labeled with a dye, such as a fluorophore, a chromophore, a radioactive moiety or a member of a specific binding pair such as biotin. The term “member of a specific binding pair” refers to a substance that specifically recognizes and interacts with a second substance exemplified by specific binding pairs such as biotin-avidin, biotin-streptavidin, antibody-antigen, and target-aptamer. Non-limiting examples of labels that can be used include fluorescent dyes such as fluorescein, fluorescein isothiocyanate, rhodamine, rhodamine isothiocyanate, Texas Red, cyanine dyes such as Cyanine 3 and Cyanine 5, and ALFXA dyes; chromophores such as horseradish peroxidase, alkaline phosphatase and digoxigenin; and radioactive moieties such as ³²P, ³⁵S, ³H, ¹²⁵I or ¹⁴C; and binding partners such as biotin and biotin derivatives. Methods for detectably labeling nucleotides, nucleotide analogs and/or primers are well-known in the art.

Nucleotides, including, but not limited to, deoxynucleotide triphosphates (dNTPs) and analogs thereof, labeled or unlabeled, can be included in primers and/or amplification reaction mixtures according to methods described herein. The term “nucleotide analog” in this context refers to a modified or non-naturally occurring nucleotide, particularly nucleotide analogs which can be polymerized, with naturally occurring nucleotides or non-naturally occurring nucleotides, by template directed nucleic acid amplification catalyzed by a nucleic acid polymerase. Nucleotide analogs are well-known in the art. Particular nucleotide analogs are capable of Watson-Crick pairing via hydrogen bonds with a complementary nucleotide and illustratively include, but are not limited to, those containing an analog of a nucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole. Nucleotide analogs include those containing an analog of a deoxyribose such as a substituted deoxyribose, a substituted or non-substituted arabinose, a substituted or non-substituted xylose, and a substituted or non-substituted pyranose. Nucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates.

Any appropriate method, illustratively including spectroscopic, optical, photochemical, biochemical, enzymatic, electrical and/or immunochemical is used to detect a label in an assay described herein.

Substrates

A solid substrate, which includes semi-solid substrate, for attachment of a capture probe can be any of various materials such as glass; plastic, such as polypropylene, polystyrene, nylon; paper; silicon; nitrocellulose; or any other material to which a nucleic acid can be attached for use in an assay. The substrate can be in any of various forms or shapes, including planar, such as silicon chips and glass plates; and three-dimensional, such as particles, microtiter plates, microtiter wells, pins, fibers and the like.

A substrate to which a capture probe is attached is encoded according to embodiments of methods and compositions of the present invention. Encoded substrates are distinguishable from each other based on a characteristic illustratively including an optical property such as color, reflective index and/or an imprinted or otherwise optically detectable pattern. For example, the substrates can be encoded using optical, chemical, physical, or electronic tags.

In particular aspects, a solid substrate to which a capture probe is attached is a particle.

Particles to which a capture probe is attached can be any solid or semi-solid particles which are stable and insoluble in use, such as under hybridization and label detection conditions. The particles can be of any shape, such as cylindrical, spherical, and so forth; size, such as microparticles and nanoparticles; composition; and have various physiochemical characteristics. The particle size or composition can be chosen so that the particle can be separated from fluid, e.g., on a filter with a particular pore size or by some other physical property, e.g., a magnetic property.

Microparticles, such as microbeads, used can have a diameter of less than one millimeter, for example, a size ranging from about 0.1 to about 1,000 micrometers in diameter, inclusive, such as about 3-25 microns in diameter, inclusive, or about 5-10 microns in diameter, inclusive. Nanoparticles, such as nanobeads used can have a diameter from about 1 nanometer (nm) to about 100,000 nm in diameter, inclusive, for example, a size ranging from about 10-1,000 nm, inclusive, or for example, a size ranging from 200-500 nm, inclusive. In certain embodiments, particles used are beads, particularly microbeads and nanobeads.

Particles are illustratively organic or inorganic particles, such as glass or metal and can be particles of a synthetic or naturally occurring polymer, such as polystyrene, polycarbonate, silicon, nylon, cellulose, agarose, dextran, and polyacrylamide. Particles are latex beads in particular embodiments.

Particles used include functional groups for attaching capture probes, such as nucleic acid capture probes, in particular embodiments. For example, particles can include carboxyl, amine, amino, carboxylate, halide, ester, alcohol, carbamide, aldehyde, chloromethyl, sulfur oxide, nitrogen oxide, epoxy and/or tosyl functional groups. Functional groups of particles, modification thereof and binding of a chemical moiety, such as a nucleic acid, thereto are known in the art, for example as described in Fitch, R. M., Polymer Colloids: A Comprehensive Introduction, Academic Press, 1997. U.S. Pat. No. 6,048,695 describes an exemplary method for attaching nucleic acid capture probes to a substrate, such as particles. In a further particular example, 1-Ethyl-3-[3-dimethylaminopropyl]carbodiimide hydrochloride, EDC or EDAC chemistry, can be used to attach nucleic acid capture probes to particles.

Particles to which a capture probe is attached are encoded particles according to embodiments of methods and compositions of the present invention. Encoded particles are distinguishable from each other based on a characteristic illustratively including an optical property such as color, reflective index and/or an imprinted or otherwise optically detectable pattern. For example, the particles can be encoded using optical, chemical, physical, or electronic tags. Encoded particles can contain or be attached to, one or more fluorophores which are distinguishable, for instance, by excitation and/or emission wavelength, emission intensity, excited state lifetime or a combination of these or other optical characteristics. Optical bar codes can be used to encode particles. The code can be embedded within the interior of the particle, or otherwise attached to the particle in a manner that is stable through hybridization and analysis.

In particular embodiments, the code is embedded, for example, within the interior of the particle, or otherwise attached to the particle in a manner that is stable through hybridization and analysis. The code can be provided by any detectable means, such as by holographic encoding, by a fluorescence property, color, shape, size, light emission, quantum dot emission and the like to identify particle and thus the capture probes immobilized thereto. In some embodiments, the code is other than one provided by a nucleic acid.

One exemplary encoded particle platform utilizes mixtures of fluorescent dyes impregnated into polymer particles as the means to identify each member of a particle set to which a specific capture probe has been immobilized. Another exemplary platform uses holographic barcodes to identify cylindrical glass particles. For example, Chandler et al. (U.S. Pat. No. 5,981,180) describes a particle-based system in which different particle types are encoded by mixtures of various proportions of two or more fluorescent dyes impregnated into polymer particles. Soini (U.S. Pat. No. 5,028,545) describes a particle-based multiplexed assay system that employs time-resolved fluorescence for particle identification. Fulwyler (U.S. Pat. No. 4,499,052) describes an exemplary method for using particle distinguished by color and/or size. U.S. Patent Application Publications 20040179267, 20040132205, 20040130786, 20040130761, 20040126875, 20040125424, and 20040075907 describe exemplary particles encoded by holographic barcodes. U.S. Pat. No. 6,916,661 describes polymeric microparticles that are associated with nanoparticles that have dyes that provide a code for the particles

While embodiments described in detail herein utilize the Luminex encoded bead platform, other types of encoded particle assay platforms may be used, such as the VeraCode beads and BeadXpress system (IIlumina Inc., San Diego Calif.), xMAP 3D (Luminex) and the like. Magnetic Luminex beads can be used which allow wash steps to be performed with plate magnets and pipetting rather than with filter plates and a vacuum manifold. Each of these platforms are typically provided as carboxyl beads but may also be configured to include a different coupling chemistry, such as amino-silane.

Particles are typically evaluated individually to detect encoding. For example, the particles can be passed through a flow cytometer. Exemplary flow cytometers include the Coulter Elite-ESP flow cytometer, or FACScan™ flow cytometer available from Beckman Coulter, Inc. (Fullerton Calif.) and the MOFLO™ flow cytometer available from Cytomation, Inc., Fort Collins, Colo. In addition to flow cytometry, a centrifuge may be used as the instrument to separate and classify the particles. A suitable system is that described in U.S. Pat. No. 5,926,387. In addition to flow cytometry and centrifugation, a free-flow electrophoresis apparatus may be used as the instrument to separate and classify the particles. A suitable system is that described in U.S. Pat. No. 4,310,408. The particles may also be placed on a surface and scanned or imaged.

Methods described herein involve use of encoded particles. The encoding of the encoded particles is used in methods described herein to identify the capture probe bound to the particles and to distinguish it from other capture probes. Each different capture probe, for example each different oligonucleotide capture probe, is associated with a different encoding of its attached encoded particle. In this way, a methylation-positive capture probe can be distinguished from a methylation-negative capture probe, and from methylation-positive and—negative capture probes for different target genomic loci. Because different encoding can distinguish the different capture probes, it is possible to use mixtures of different capture probes when assaying a single DNA sample in a multiplexed format.

Binding of Capture Probes to Substrate

Association of the capture probes with a substrate is achieved by any of various methods, illustratively including adsorption and chemical bonding. Binding of the nucleic acid capture probes to a substrate is achieved by any of various methods effective to bond a nucleic acid to a solid or semi-solid substrate, illustratively including adsorption and chemical bonding. The nucleic acids can be bonded directly to the material of the encoded particles or indirectly bonded to the encoded particles, for example, via bonding to a coating or linker disposed on the particles. Nucleic acids can be synthesized, and/or modified once synthesized, to include a functional group for use in bonding the nucleic acids to particles. For example, the nucleic acids sequences used as probes can include carboxyl, amine, amino, carboxylate, halide, ester, alcohol, carbamide, aldehyde, chloromethyl, sulfur oxide, nitrogen oxide, epoxy and/or tosyl functional groups. In the methods described herein, the capture probes can be in physical association with the substrate prior to contacting with labeled amplified converted DNA. Alternatively, the capture probes can be contacted with labeled amplified converted DNA and then be brought into physical association with the substrate, for example, by binding of a tag on the capture probe with a tag-binding partner on the substrate.

In particular embodiments of assays described herein, amplified converted DNA is captured by the capture probes attached to the encoded particles by hybridization.

Provided are assays according to embodiments of the present invention using more than one type of encoded particles. In particular embodiments, a “particle set” is provided wherein each particle of the particle set is encoded with the same code such that each particle of a particle set is distinguishable from each particle of another “particle set.” In further embodiments, two or more codes can be used for a single particle set. Each particle can include a unique code, for example. In certain embodiments, particle encoding includes a code other than or in addition to, association of a particle and a nucleic acid capture probe specific for a target nucleic acid.

Multiplex Methods and Compositions

Methods are provided herein for assay of methylation state of a genomic locus in combination with assay of a second characteristic of the sample nucleic acids. Assay of a second characteristic can be any of a variety of characteristics illustratively including, but not limited to, assay of the methylation status of a second target genomic locus and assay of a gender-specific genomic locus to determine gender of the subject from whom the genomic DNA was obtained.

The methods described herein can be performed in a multiplexed format. For example, probes such as oligonucleotide capture probes can be designed to bind to protected and unprotected PCR products for a first target genomic locus, each probe bound to its uniquely identifying particle. For a second target genomic locus, probes can be designed to bind to protected and unprotected PCR products for the second target genomic locus, each probe bound to its uniquely identifying particle. This type of assay design would be repeated for each target genomic locus of interest, up to the limit of the particular encoded particle system used. For example, the Luminex system used in the Example below is presently limited to 100 different unique encodings. Other encoded particle assay platforms accommodate higher multiplexing as well. PCR multiplexing of between 6 and 10 regions is routine, easily supporting panels of disorders characterized by methylation of CpG islands. Thus, detection of the methylation status of two or more, three or more, ten or more, and even hundreds of target genomic regions is envisioned using the methods described herein.

Accordingly, provided herein is method for detecting methylation within two or more target genomic loci containing one or more CpG islands. The method involves treating independently each DNA sample containing a respective target genomic locus with bisulfite to convert unmethylated cytosine to uracil; amplifying a segment of each treated DNA using primers that flank the respective target genomic locus, wherein an unprotected PCR product is produced when the target genomic locus lacked methylation, and a protected PCR product is produced when the target genomic locus contained methylation; contacting resultant amplified segments with, for each target genomic locus, (i) an oligonucleotide probe selective for the unprotected PCR product, the probe being attached to a methylation-negative encoded particle unique to a particular target genomic locus, and (ii) an oligonucleotide probe selective for the protected PCR product, the probe being attached to a methylation-positive encoded particle unique to a particular target genomic locus, such that a complex of PCR product hybridized to a respective oligonucleotide probe attached to an encoded particle can produce a signal, detecting signals associated with the methylation-negative and methylation-positive encoded particles, thereby detecting the presence or absence of methylation within each of the target genomic loci.

Assay of a gender-specific genomic locus includes assay of an amelogenin gene according to some embodiments.

Thus, provided herein is a method for simultaneously determining gender and DNA methylation state. The method involves providing a DNA sample obtained from a subject, the DNA comprising a target genomic locus comprising one or more CpG islands; treating the DNA sample with bisulfite to convert unmethylated cytosine to uracil; amplifying a segment of the treated DNA using primers that flank the target genomic locus, wherein an unprotected PCR product is produced when the target genomic locus lacked methylation, and a protected PCR product is produced when the target genomic locus contained methylation; amplifying a segment of the treated DNA using primers specific for an amelogenin sequence common to male and female subjects, wherein a segment of the amelogenin sequence that varies between male and female subjects is amplified; contacting resultant amplified segments with (i) an oligonucleotide probe selective for the unprotected PCR product, the probe being attached to a methylation-negative encoded particle, (ii) an oligonucleotide probe selective for the protected PCR product, the probe being attached to a methylation-positive encoded particle, (iii) an oligonucleotide probe selective for a male-specific amelogenin sequence, the probe being attached to a male-positive encoded particle; and (iv) an oligonucleotide probe selective for a female-specific amelogenin sequence, the probe selective for a female-specific amelogenin sequence, such that a complex of PCR product hybridized to a respective oligonucleotide probe attached to an encoded particle can produce a signal, and detecting signals associated with the methylation-negative encoded particles, methylation-positive encoded particles, male-positive encoded particles and female-positive encoded particles, thereby detecting the presence or absence of methylation within the target genomic locus and the gender of the subject.

Methods described can be performed in any suitable container. In particular embodiments, for example, where multiple samples are to be assayed, a multi-chamber container can be used. Multi-chamber containers illustratively include multi-depression substrates such as slides, silicon chips or trays. In some embodiments, each sample is disposed in a different well of a multi-well plate. For example, a multi-well plate can be a 96-well, 384-well, 864-well or 1536-well assay plate.

Kits for detecting the methylation status of a target genomic locus are provided. In particular embodiments, a kit is provided which includes an encoded particle set and/or a mixture of two or more encoded particle sets, wherein each particle set includes attached capture probes specific for a target nucleic acid. Instructional material for use of the encoded particle set and/or multiplex reagent including two or more encoded particle sets is optionally included in a kit. An ancillary reagent such as buffers, enzymes, washing solutions, hybridization solutions, detectable labels, detection reagents and the like are also optionally included.

Exemplary assays described herein demonstrate specific detection of methylated and unmethylated DNA in a CpG island promoter region, as well as combining such detection with a vender determination.

Embodiments of inventive compositions and methods are illustrated in the following examples. These examples are provided for illustrative purposes and are not considered limitations on the scope of inventive compositions and methods.

### Example 1

This example describes assays performed using the Fragile X syndrome promoter locus as a representative target genomic locus. In X chromosome-linked disorders such as Fragile X it is useful to assess the relative amounts of both the unmethylated and the methylated forms of genomic DNA in the promoter region, as males and females have a matrix of possible states due to their different numbers of X chromsosomes. Normal females have 2 copies of the X chromosome, and one of them is methylated in normal subjects as a consequence of a process called X chromosome inactivation. Males have only one X chromosome, and the promoter region is not methylated in a normal subject and is methylated in an affected subject. A simplified representation of this is shown in the table below.

| | Male subject | | Female subject | |
|---|---|---|---|---|
| | Methylated | Unmethylated | Methylated | Unmethylated |
| Normal | 0 | 1 | 1 | 1 |
| Affected | 1 | 0 | 2 | 0 |

Intrepretation of the methylation state can be complicated when the affected subject presents a mosaic cellular case. For example, affected mosaic males can have both methylated and unmethylated states detected. Without an additional assay to determine gender, this subject could be diagnosed as a normal female, when actual gender is unknown. Additionally, a failed PCR or assay on a female subject resulting in detection of only the methylated state could be misdiagnosed as an affected male without a confirming gender assay. In a non-mosaic case, if one were to test only for the unmethylated state, without knowledge of the methylated state, a failed PCR or failed assay would be indistinguishable from an affected male or female. Conversely, if one were to test only for the methylated state, without knowledge of the unmethylated state, an affected male and an unaffected female would give approximately the same signal, requiring the availability of the subject's sex information in order to interpret the results.

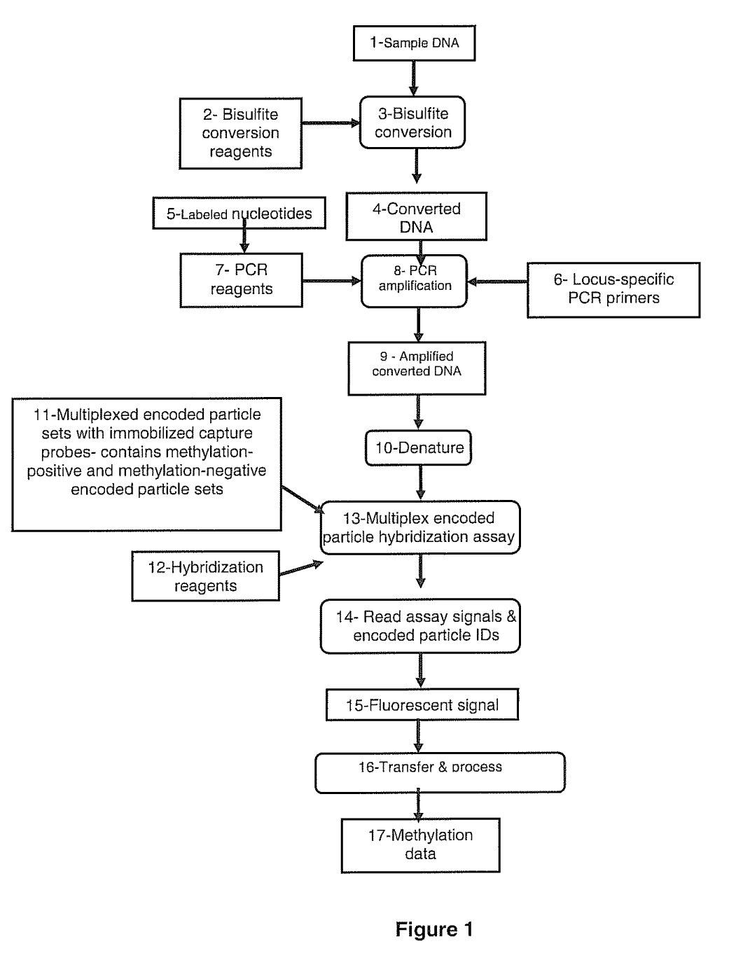

FIG. 1 shows an exemplary process for carrying out the methods described herein. First, the sample DNA 1 is the input to the assay. The DNA is then sodium bisulfite converted 3, a process that chemically changes the sequence of unmethylated DNA while preserving the sequence of “protected” methylated DNA utilizing bisulfite conversion reagents 2. This is a well-known procedure, in this example performed with a kit, the EZ-DNA Methylation Gold kit (Zymo Research, Orange Calif.). Bisulfite treatment converts unmethylated C (cytosine) nucleotides to U (uracil) while methylated C nucleotides remain unconverted. The kit includes components for performing a standard column purification process to clean up the bisulfite treated DNA from the residual conversion chemicals and byproducts. The product of the bisulfite treatment, whether any nucleotides have actually been converted or not, is referred to as converted DNA 4. Converted DNA that had contained methylation is sometimes referred to herein as a “protected amplification product” or “protected PCR product,” whereas converted DNA that did not contain methylation is sometimes referred to herein as an “unprotected amplification product” or “unprotected PCR product.”

The converted DNA 4 is then subjected to PCR amplification 8 using primers 6 and PCR reagents 7. In this example the PCR reagents comprised the Hot Start Taq polymerase enzyme and buffer (Qiagen Inc. USA, Valencia Calif.) and a 0.18 mM dNTP nucleotide mix (Invitrogen, Carlsbad Calif.) in which 50% of the d-CTP nucleotides 5 were biotinylated (PerkinElmer, Boston Mass.). The PCR reagents were:

Hot Star Taq DNA polymerase (Qiagen) 1.25 units

1× PCR buffer (1.5 mM MgCl₂+salts) (Qiagen)

180 μM dGTP (Invitrogen)

180 μM dATP (Invitrogen)

180 μM dTTP (Invitrogen)

90 μM dCTP (Invitrogen)

90 μM biotin-11-dCTP (PerkinElmer)

The PCR reactions were 50 μl.

Locus-specific primers 6 were designed for the target locus of the assay, the Fragile X gene promoter locus in this example. Specifically, primers were designed to straddle a region of the CpG island, within the promoter of the targeted gene, that is either methylated or not depending on the regulation state of the individual.

One forward PCR primer and two different reverse primers were used in this example, all at 500 nM in the PCR reaction. The forward primer is specific to the genomic locus but non-specific to the pre-conversion methylation state of the sample DNA. Each of the two reverse primers was designed for converted methylated or unmethylated DNA respectively. The primer sequences were:

| | |
|---|---|
| Forward: | |
| GGG GTC GAG GGG TTG AGT | (SEQ ID No. 1) |
| Reverse 1: | |
| ACC GCA CGC CCC CTA ACA | (SEQ ID No. 2) |
| Reverse 2: | |
| ACC ACA CAC CCC CTA ACA | (SEQ ID No. 3) |

In this example, the PCR amplification 8 was driven by thermal cycling, using an MJ Research PTC 100 (Watertown Mass.) instrument. The cycles were:

95° C. for 15 minutes to activate the polymerase

Then the following temperature sequence:

95° C. melting, 30 sec

57° C. annealing, 60 sec

72° C. extension, 60 sec

for 40 cycles to produce amplified converted DNA 9.

The PCR converted any U (uracil) nucleotides in the amplified converted DNA 8 to T (thymine) nucleotides. The PCR products generated by these primers are 222 bases long.

The amplified converted DNA 9 was then denatured 10 by heating to 98° C. to prepare it for the subsequent encoded particle hybridization assay 13, wherein multiplexed encoded particle sets with immobilized capture probes 11 and hybridization reagents 12 were inputs into the hybridization assay 13. In this example the encoded particles are □5 μm diameter polystyrene beads encoded with impregnated mixtures of fluorescent dyes. The example hybridization assay is described in detail with further figures below. In this example, after hybridization the multiplex encoded particle assay was read 14 on the Luminex xMAP platform (Luminex, Austin Tex.). The results of the assay are fluorescent signals recorded in a fluorescent signal data file 15. This file is then transferred 16 to another program or computer for subsequent analysis to determine the methylation status of each sample from the fluorescent signal data. The methylation state data 17 for each targeted locus for each sample is the final result.

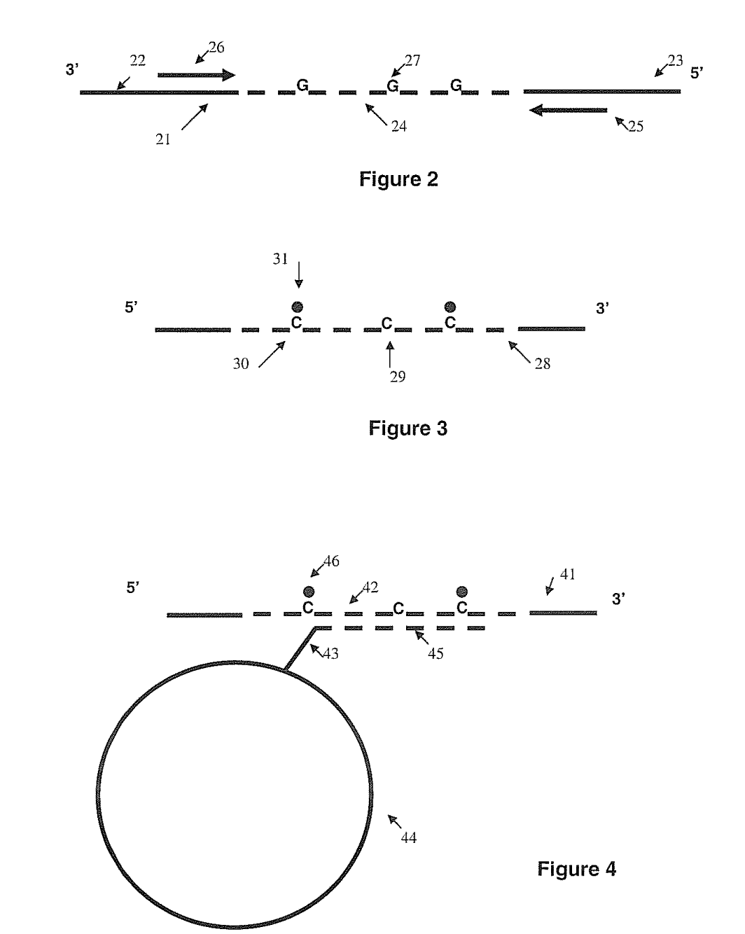

FIG. 2 depicts schematically the PCR design for another method for determining the methylation state of a DNA sequence containing a CpG island. The target sequence 21 is characterized by 3 zones. The CpG-rich zone 24 of the promoter region of the target gene is where the methylation regulatory mechanism is most densely applied. The CpG-rich sequence is flanked on both ends by flanking sequences 22 and 23 that may or may not contain some CpG dinucleotides. PCR primers 25 and 26 are designed to be complementary to the flanking sequences and to prime amplification across the CpG-rich sequence. The forward primer 26 was simple and the reverse primers 25 were a composite pair.

Wherever the template DNA 24 has G nucleotides 27, the resulting PCR product strand has C nucleotides. As the PCR nucleotide mix utilizes 50% biotin labeled dCTP nucleotides approximately 50% of the C nucleotides in the PCR product are biotin labeled. This is depicted schematically in FIG. 3, where the PCR product molecule 28 produced from the template in FIG. 2 has C nucleotides 30 at the loci of the Gs in the template. A fraction of these, approximately 50%, are labeled nucleotides incorporating a biotin 31.

The PCR product molecule's central portion 29 between the primer loci, the converted and amplified CpG-rich sequence, now has a different sequence depending on whether the template DNA in the original sample was methylated or not. If the sample were methylated, all Cs in the CpG dinucleotides remain Cs. If it were unmethylated the Cs would have been amplified as Ts. In addition to the sequence transformation the PCR process amplifies the target DNA amount by a factor of approximately 1,000 to 100,000, simplifying detection in the subsequent hybridization assay. This significant sequence difference in the PCR products is useful for detection using a hybridization assay.

FIG. 4 is a schematic depiction of specific capture of PCR products 41 to an oligonucleotide probe 43 that has been immobilized to an encoded particle 44, such as a Luminex bead. The figure is schematic only; it is out of scale and shows only one immobilized oligonucleotide probe molecule whereas typically hundreds, thousands or millions of capture probe molecules are present on each particle or bead. The capture probe 43 is depicted as immobilized at the amino end of the oligonucleotide. The sequence on the other end 45 of the capture probe is designed to be complementary to the methylation-state-specific sequence portion 42 of the converted DNA PCR product. A bead or encoded particle is coupled to only one methylation specific capture probe sequence; each bead of the pair thus captures only one of the products from either methylated or unmethylated samples.

Each captured PCR product molecule 41 is labeled with biotin 46 on about 50% of the C nucleotides in the sequence. Biotin is one of many types of labels suitable for the methods described herein. The length of the capture region of the probes and target PCR product was 50 nucleotides in this example, although other lengths from about 20 to about 100 nucleotides can be used.

In this example the capture probe sequence for methylated converted amplified DNA was:

(SEQ ID No. 4)
CGCCTCCGTCACCGCCGCCGCCCGCGCTGGCCGTCGACCCGCCGCCCGCT

The capture probe sequence for unmethylated converted amplified DNA was:

(SEQ ID No. 5)
CACCTCCATCACCACCACCACCCACACTCACCATCAACCCACCACCCACT

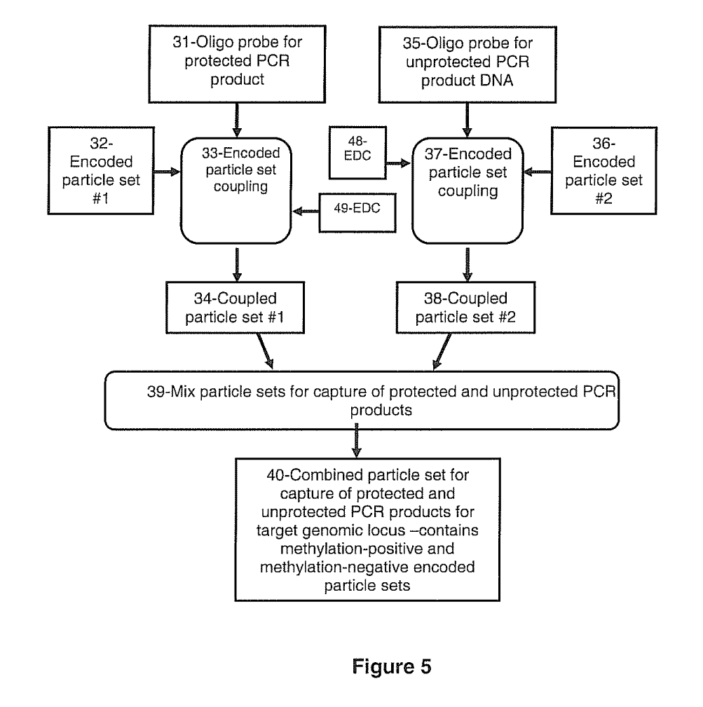

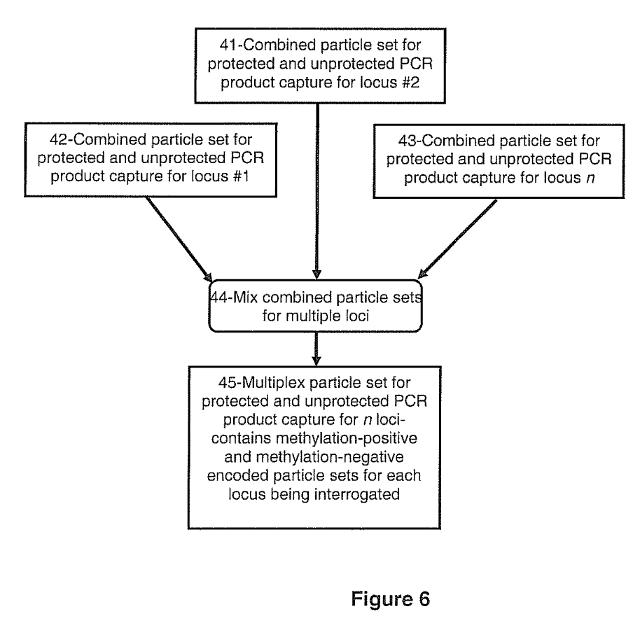

FIG. 5 shows a schematic process flowchart showing one method for coupling oligonucleotide capture probes 31 and 35, to encoded particle sets 32 and 36, then mixing the sets to form a combined bead set.40. Oligonucleotide capture probes 31 complementary to the converted methylated-specific portion of the sequence of the above-described PCR products were designed and synthesized. Also, oligonucleotide capture probes 35 complementary to the post-conversion unmethylated-specific portion of the sequences of the above-described PCR products were designed and synthesized. In this example, said probe oligonucleotides were synthesized with a terminal amine group to facilitate end-coupling to a carboxylated Luminex bead. The coupling was performed by incubating the capture probes 31, 35, individually with the encoded bead sets 32 and 36 with EDC (1-ethyl-3-(3-dimethylaminopropyl)carbodiimide) 48, 49 in MES buffer according to a standard protocol (Luminex). The oligo-coupled bead sets 34, 38 were then mixed to form a combined bead set 40. The depicted process is used for making a 2-plex combined bead set for capturing the converted amplified products of methylated and unmethylated sample DNA from single genomic locus (such as the CpG island associated with Fragile X). Pairs of probes for detecting the methylation state of other loci can also be prepared, coupled to additional bead sets and mixed into a larger multiplex bead set as illustrated in the schematic process flowchart of FIG. 6. A combined bead set 42 for region #1 is mixed 44 with combined bead sets 42 for locus #2 and so on up to a bead set 43 for locus n. For example, a CpG island associated with the Prader-Willi/Angleman syndrome locus could be used simultaneously with the bead set for the Fragile X locus of this example, to create a 4-plex hybridization assay that would evaluate the methylation state of 2 separate loci.

If desired, additional probes can be added to the multiplex assay to act as internal controls, so as to normalize output signal values. As an example, the XIST gene is methylated in normal females by a process called X chromosome inactivation. This methylation mechanism is independent of the methylation of the FMR promoter caused by CGG trunucleotide expansion of the Fragile X syndrome and therefore can be used as a control to normalize the signal. Internal controls can be devised for other loci, disorders, or indicators as well.

Encoded particle assay platforms can support the simultaneous assaying of several genomic loci. The first-generation Luminex encoded bead platform supports hybridization multiplexing up to 100-plex assays, for example. Testing for a panel of multiple disorders at one time can have significant cost advantages, as most of the costs of the assay are fixed regardless of the “plex” or number of targets.

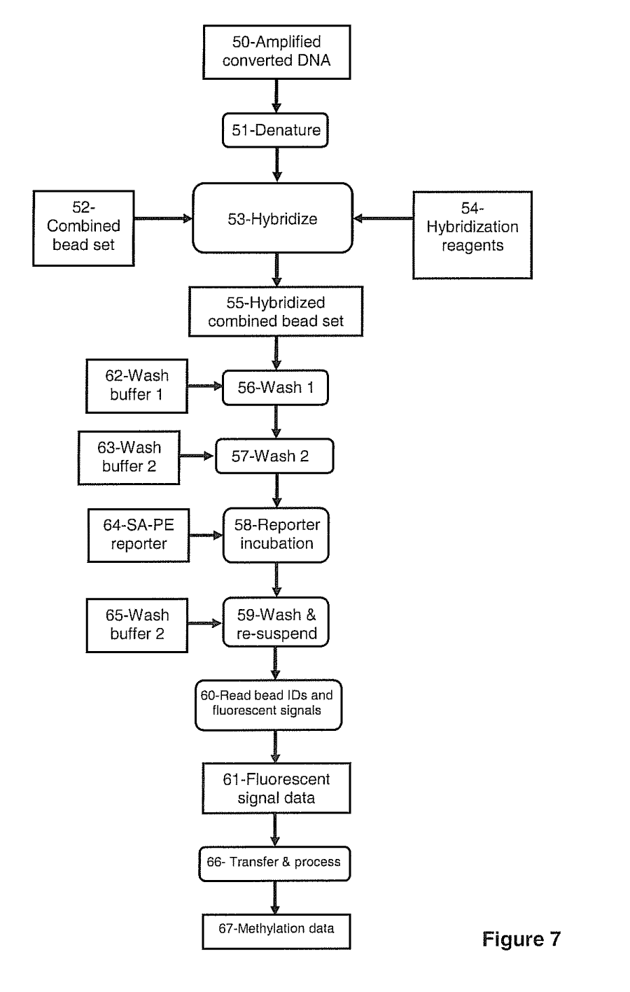

FIG. 7 is a schematic process flow diagram of an exemplary multiplex hybridization assay. It shows in greater detail the processes described by elements 9 through 17 in FIG. 1. Referring to FIG. 7 the input to the hybridization assay is amplified converted DNA 50 as described above. It is denatured 51 prior to hybridization, in this example by heating for 5 minutes to a temperature of 98° C., then holding the DNA at 50° C. until transferring it into the hybridization reaction 53. A combined bead set 52 for the assay, containing the combined unmethylated and methylated capture probe bead set described above, and hybridization reagents 54 were mixed together in an incubation vessel, such as the well of a 96-well hard-shell PCR microplate (Bio-Rad Laboratories, Hercules Calif.).

| | |
|---|---|
| Hybridization buffer, 1.5x TMAC | 33 μl |
| Amplified converted DNA sample | 17 μl |
| | 50 μl hybridization volume |

The hybridization reagents in this example were a 1.5× TMAC buffer as recommended by Luminex. The plate was sealed with a foil sealer (Bio-Rad) and hybridization 53 was performed via a 50° C. incubation while shaking at 1,200 RPM for 15 minutes in a TriNest microplate shaking incubator (PerkinElmer, Turku FI).

Completion of the hybridization 53 produces a hybridized combined bead set 55, wherein each PCR product captured onto the beads by the immobilized probes has been labeled with biotins. The hybridized bead set 55 was then washed by transferring the beads by pipette to a filter plate (Millipore, Bedford Mass.). The hybridization buffer was aspirated from the filter plate with a microplate vacuum manifold (Millipore) at 5″ Hg vacuum for about 5 seconds. A first wash was done by adding 100 μl of Wash buffer 1 62 containing 2×SSC buffer with 50% formamide to each well of the filter plate, mixing with the pipette, then incubating the non-sealed filter plate for 5 minutes at 50° C. and 1,200 rpm. The wash buffer 1 was then vacuum aspirated and 100 μl of wash buffer 2 63 was added to each well, followed by vacuum aspiration. This second wash was performed twice as the Wash 2 process 57. In the example assay, Wash buffer 2 was a detergent wash, 1× PBS with 0.01% Tween-20.

In the example assay, a streptavidin-phycoerythrin reporter 64 (Prozyme, San Leandro Calif.) is first diluted 1:250 by 1× PBS with 0.1% BSA and 0.05% Tween-20. 75 μl of the diluted reporter 64 was then incubated 58 with the washed hybridized bead set. Phycoerythrin is the commonly used reporter for use with the Luminex detection platform but other fluorescent or non-fluorescent reporters could be used in other situations. The reporter was incubated 58 in the non-sealed filter plate for 5 minutes at 50° C., shaking at 1,200 RPM. After the reporter incubation the beads were washed and resuspended 59 in 100 μl and 75 μl respectively of the wash buffer 2 65 used in the previous wash step. The bead set was then read 60 in a detection instrument suitable for the encoded multiplex particle type, the Luminex 200 instrument in this example. The instrument produced fluorescent signal data file 61, where the fluorescent intensity values are indicative of the number of labeled converted PCR product targets captured on each bead set. This fluorescent signal data was then transferred 66 in the form of a text file to downstream analysis software which calculated data 67 indicative of the methylation state of each sample at the interrogated locus or loci.

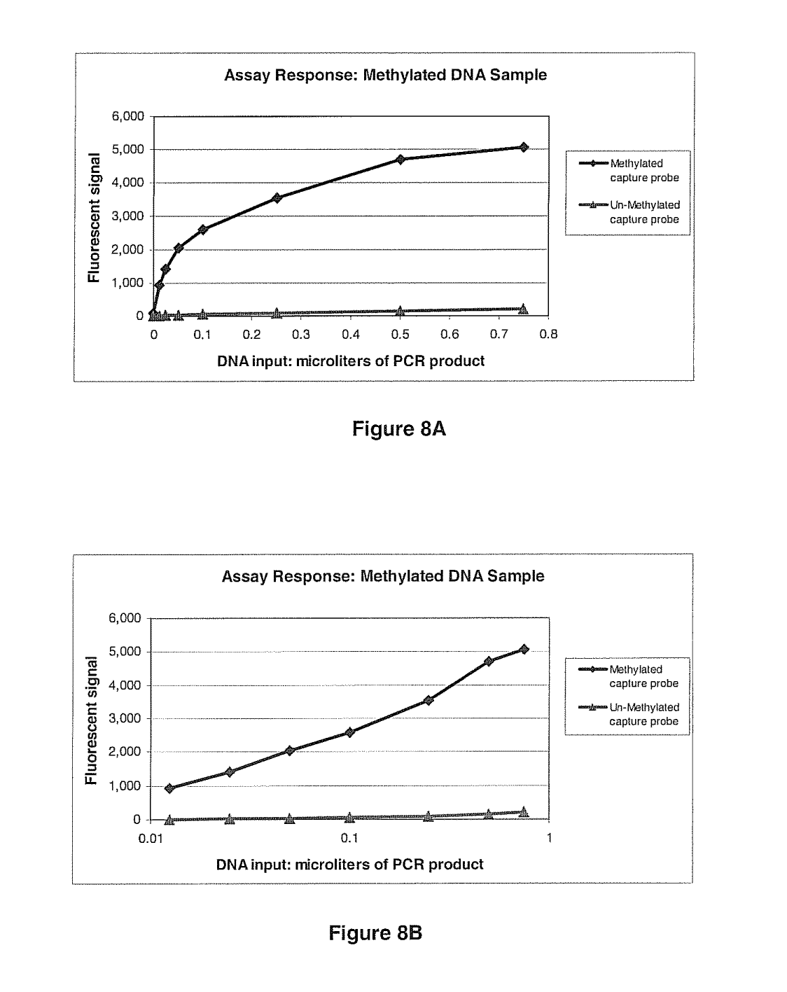

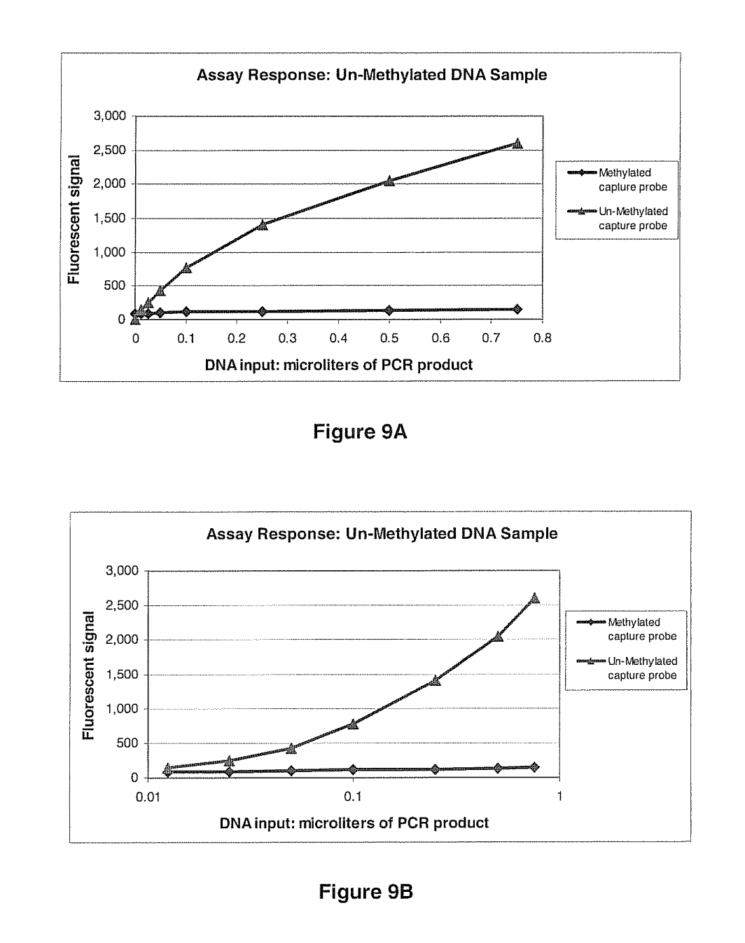

FIGS. 8 and 9 show graphical results from the example assay. PCR primers and oligonucleotide capture probes were designed as described above for the CpG island associated with the Fragile X locus. The samples for this example assay had known methylation states. Both unmethylated and methylated samples were EpiTect PCR Control DNA (Qiagen). The methylated EpiTect DNA has been fully methylated enzymatically under controlled conditions. Each of these reference samples was assayed according to the exemplary method of the present invention described above. A description of the nomenclature used and the data shown in FIGS. 8 and 9 is as follows.

All four FIGS. 8a, 8b, 9a, and 9b show dose response curves of the example assay. To vary the input amounts of converted amplified DNA generated from unmethylated and methylated samples the PCR products were serially diluted. The amount of the PCR product in each 17 μl dilution used input to the hybridization is shown on the horizontal axis of each plot. Note that the highest input amount for which results are shown is 0.75 microliters (from the 50 HCl PCR reaction) beyond which the assay saturated. This indicates that a smaller PCR reaction, lower number of PCR cycles, or both would be appropriate simplifications for future assays.

The vertical axis of the plots of all four FIGS. 8a, 8b, 9a, and 9b is the fluorescent signal for each input amount as reported by the Luminex reading instrument. This value is reported as MFI, for Median Fluorescent Intensity, in the Luminex data files. In each plot the signals from the beads with the capture probe for methylated converted amplified DNA from the target locus are denoted with diamonds, while the signals from the beads with the capture probe for unmethylated converted amplified DNA from the target locus are denoted with triangles.

Referring to FIG. 8a, the data shown is for the example assay run on methylated reference DNA. The signals produced by the capture probes for the product of methylated sample show a monotonically increasing dose response of fluorescent signals, from near zero signal at zero input DNA to about 5,000 MFI at 0.75 μl input of the PCR reaction. The signals produced by the capture probes for the product of unmethylated sample remain near zero, at less than 250 MFI, throughout the range of input amounts. This demonstrates sensitivity and specificity of the detection of a methylated sample in the example assay.

FIG. 8b depicts the same data as FIG. 8a except the horizontal (DNA input amount) axis has been represented on a logarithmic scale.

FIG. 9 shows data from the example assay run on unmethylated reference DNA. The signals produced by the capture probes for the product of unmethylated sample show a monotonically increasing dose response of fluorescent signals, from near zero signal at zero input DNA to about 2,500 MFI at 0.75 μl input of the PCR reaction. The signals produced by the capture probes for the product of methylated sample remain near zero, at less than 150 MFI, throughout the range of input amounts. This demonstrates sensitivity and specificity of the detection of an unmethylated sample in the example assay.

FIG. 9b depicts that same data as FIG. 9a except the horizontal (DNA input amount) axis has been represented on a logarithmic scale.

### Example 2

This example describes assays performed using the Fragile X syndrome promoter locus as a representative target genomic locus, using samples collected from affected and unaffected individuals.

The experimental work was performed essentially as is described in Example 1, with variations as described below.

For the PCR amplification, 35 rather than 40 cycles were performed. PCR conditions can be varied, depending on the selected enzyme, buffers, reaction components, PCR machines and the like.

The capture probe sequence for methylated converted amplified DNA was: CGC CTC CGT CAC CGC CGC CGC CCG CGC TCG CCG TCG A (SEQ ID No. 6)

The capture probe sequence for unmethylated converted amplified DNA was: TTG GTT TTA TTT TTG GTG GAG GGT TGT TTT TGA GTG GGT G (SEQ ID No. 7)

In this Example, referring to FIG. 7, the input to the hybridization assay was heated for 5 minutes to a temperature of 98° C., then holding the DNA at 60° C. until transferring it into the hybridization reaction.

In this Example, the hybridized bead set 55 was incubated further for 5 minutes at 50° C. and 1,200 rpm, after the addition of 50 μl of Wash buffer 1 62 comprising 2×SSC buffer with 50% formamide to each well of the hard shell plate. After the incubation with Wash buffer 1, the washed beads were transferred by pipette to a filter plate (Millipore, Bedford Mass.). The Wash/hybridization buffer was aspirated from the filter plate with a microplate vacuum manifold (Millipore) at 5″ Hg vacuum for about 5 seconds. A 100 μl volume of Wash buffer 2 63 was added to each well, followed by vacuum aspiration. This wash was performed twice as the Wash 2 process 57. In the example assay, Wash buffer 2 was a detergent wash, 1× PBS with 0.01% Tween-20.

Referring to 58, the reporter was incubated in the non-sealed filter plate for 5 minutes at 37° C., rather than 50° C., shaking at 1,200 RPM.

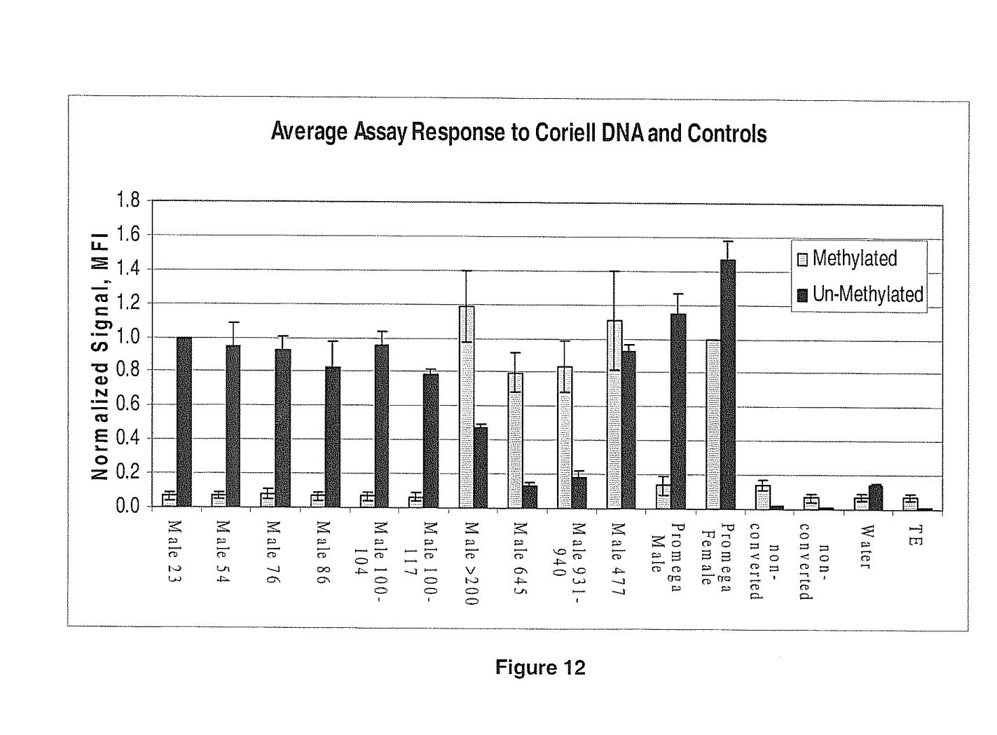

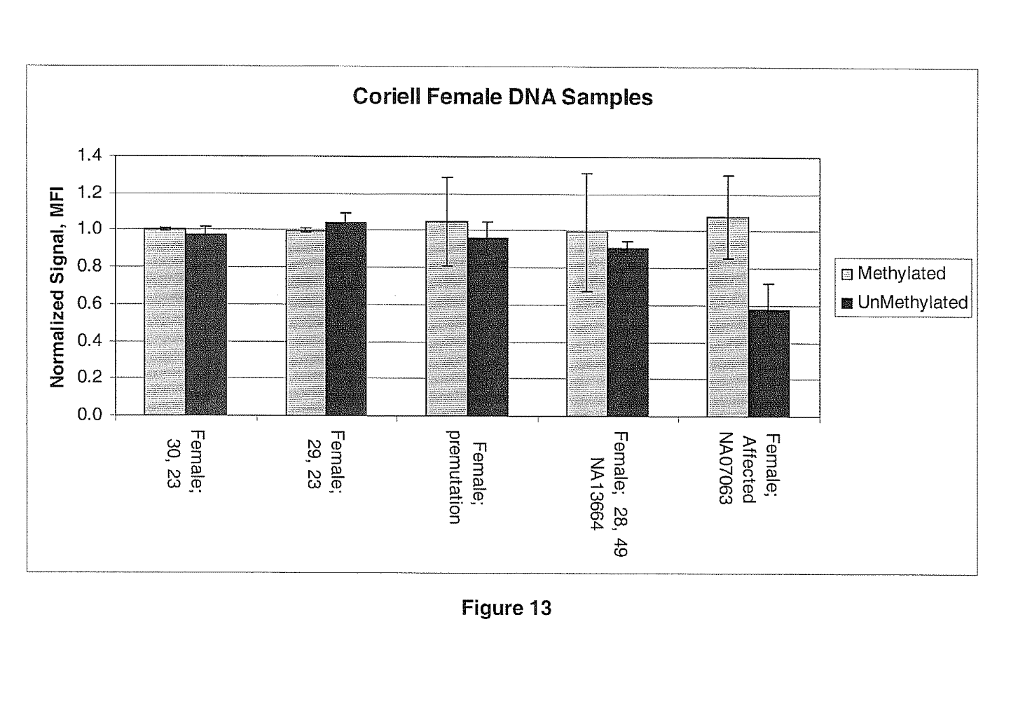

FIGS. 12 and 13 show graphical results from these experiments. For each graph, the vertical axis is the fluorescent signal for each of the samples as reported by the Luminex reading instrument as Median Fluorescent Intensity (MIFI). The signals from the beads with the capture probe for methylated DNA from the target locus are shown on the left side of the pair-wise signals for each sample, while the signals from the beads with the capture probe for unmethylated DNA from the target locus are shown on the right side of the pair-wise signals for each sample.

Referring to FIG. 12, the data shown is for the example assay run on human male samples. The samples were obtained from Fragile X affected males (Male>200; Male 645; Male 931-940 and Male 477, the numbers referring to the number of CGG repeats) and unaffected males (Male 23, Male 54, Male 76, Male 86; Male 100-104 and Male 100-117). Control samples included control unaffected male (Promega Male); control unaffected female (Promega Female); samples that did not undergo bisulfite treatment (non-converted); and nucleic acid-free samples (Water and TE). As can be seen, the methylation-positive signal was increased in affected males, in comparison with unaffected males, while the methylation-negative signal was decreased in affected males, in comparison to unaffected males.

Referring to FIG. 13, the data shown is for the example assay run on human female samples. The samples were obtained from Fragile X affected females (NA13664 and NA07063), and unaffected females (Female 30, 23; and Female 29, 23) and Female 30, >200). As can be seen, the methylation-positive signal was increased in affected females, in comparison with unaffected females, while the methylation-negative signal was decreased in one affected female, in comparison to unaffected females.

### Example 3

This example describes a method to determine both the Fragile X methylation state and the gender of a bisulfite converted DNA sample in a multiplex assay format. Gender determination is useful, for example, for clarifying the results obtained from the methylation assay for Fragile X in some cases. Specifically, the interpretation of the methylation results from an affected mosaic male would be complicated by the detection of both methylated and unmethylated states. Without an assay to determine gender, this subject could be diagnosed as an unaffected female, when gender is unknown.

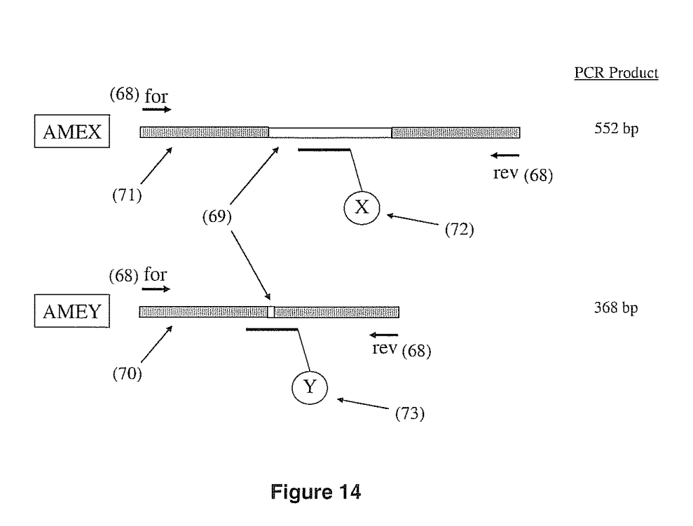

Currently the most common assay to determine gender is the amelogenin test. Amelogenin protein is transcribed from genes found on both chromosomes X and Y. These genes (AMEX and AMEY) are largely homologous, but have regions of sequence variance that have been used in PCR based assays to detect the presence of either AMEX or AMEY and thereby define gender. For example as shown in FIG. 14, a widely used method has a single pair of primers (68) complimentary to sequence shared by both AMEX and AMEY, but amplifies across a variant sequence region (69). The resulting PCR products vary in size and the different alleles are identified by capillary or agarose electrophoresis. An embodiment of this amelogenin test has been adapted for gender determination in this exemplary assay described herein by using primers that will hybridize to DNA modified by bisulfite conversion. The PCR product for Y (70) is smaller in size, as it has missing sequence unique to the PCR product for X (71). The unique sequence found in the PCR product for X can be hybridized to an oligonucleotide probe that is associated with an encoded particle (72). Additionally, a separate oligonucleotide associated with a differently encoded particle (73) can be hybridized to sequence unique to the PCR product for Y. In this way, an X-positive probe can be distinguished from a Y-positive probe, and determine the gender of the subject. Because different encoding can distinguish the different oligonucleotide probes, it is possible to mix encoded particle/probe sets to detect gender in addition to methylation signatures from a DNA sample. This can be done by mixing the amelogenin bisulfite PCR products with the Fragile X bisulfite PCR products together in a hybridization reaction with encoded particle/probe sets for both gender determination and the methylation signature of the FMR promoter region. Additionally, the PCR can be done in a multiplex format to simultaneously amplify both targets prior to hybridization

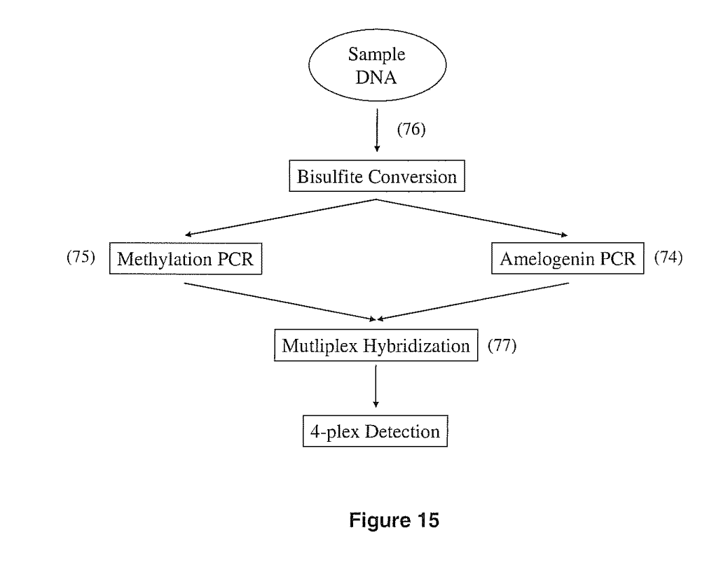

In this example, as shown in FIG. 15, the methylation state and gender of clinical samples was determined by mixing the amelogenin bisulfite PCR products (74) with the Fragile X bisulfite PCR products (75) together in a hybridization reaction with a four-plex encoded bead/probe combination which can identify both sample gender and the sample methylation signature of the FMR promoter region. The experimental work for bisulfite conversion of sample DNA (76) and the PCR amplification of the FMR promoter region was performed as is described in Example 2. The experimental work to multiplex the hybridization of the PCR amplified converted FMR DNA with the PCR amplified converted amelogenin DNA was performed essentially as described in Example 2 with variations as described below.

Referring to FIG. 14, locus-specific primers were designed for the amelogenin gene target. Specifically, primers were designed to anneal to complimentary sequence shared by both AMEX and AMEY, and to be extended by PCR across the sequence region that is different between the two genes. Additionally, the primers were also designed to anneal to bisulfite converted DNA, so as to be used concurrently on the bisulfite converted DNA prepared for the Fragile X methylation specific test. This exemplary gender detection assay does not require bisulphite treatment, but is done in this example to conserve sample DNA. Primers could also be designed for DNA not treated by bisulfite as the amelogenin genes are not methylation regulated and do not have CpG islands.

The amelogenin specific PCR conditions were essentially as described in Example 1 with the following exceptions. A forward PCR primer was used at 300 nM and a reverse primer was used at 700 nM in the PCR reaction for this example. The primer sequences were:

(SEQ ID No. 8)
Forward:
GCG GCC ACC TTA CTC ATA TTA TAC TTA ACA
(SEQ ID No. 9)
Reverse:
GCG GCG TTG ATG GTT GGT TTT AAG TTT GTG

In this example, the PCR cycles for the Amelogenin target were:

95° C. for 15 minutes to activate the polymerase

Then the following temperature sequence:

94° C. melting, 1 minute

55° C. annealing, 90 sec

72° C. extension, 90 sec

for 40 cycles to produce amplified converted DNA.

The capture probe sequence for the AMEX specific bisulphite converted amplified DNA was: GTT TTT GTT GAA ATA TTA GTG ATT AAG TGG TAT AGG AGA G (SEQ ID No. 10)

The capture probe sequence for the AMEY specific bisulphite converted amplified DNA was: GGT AAA ATG TAA AGA TTA GGT AAA ATT ATT AAT TTT GGG TAA ATA ATT TT (SEQ ID No. 11)

In this Example, referring to FIG. 15, the input amount of amplified converted DNA added to the hybridization assay was composed of a mixture of bisulphite converted amplified PCR DNA. This was done by mixing 0.1 μL of the amelogenin PCR DNA (74), with 1 μL of the FMR promoter region PCR DNA (75) and buffer {10mM Tris-HCL (pH 8.0), 1mM EDTA} to a volume of 17 μL. This mixture of converted amplified PCR DNA for both amelogenin and FMR was combined with 33 μL of hybridization mix that contained four different beads sets each with a different immobilized probe (77). As described in Example 2, one encoded bead/probe set had the immobilized capture probe sequence specific for the methylated converted amplified DNA and a second encoded bead/probe set had the immobilized capture probe sequence for unmethylated converted amplified DNA. The other two encoded bead/probe sets had the immobilized capture probe sequence for either the AMEX or the AMEY described above. The four-plex DNA hybridization, wash steps and detection of the biotin labeled product captured onto the beads by the immobilized probes was done as described in Example 2.

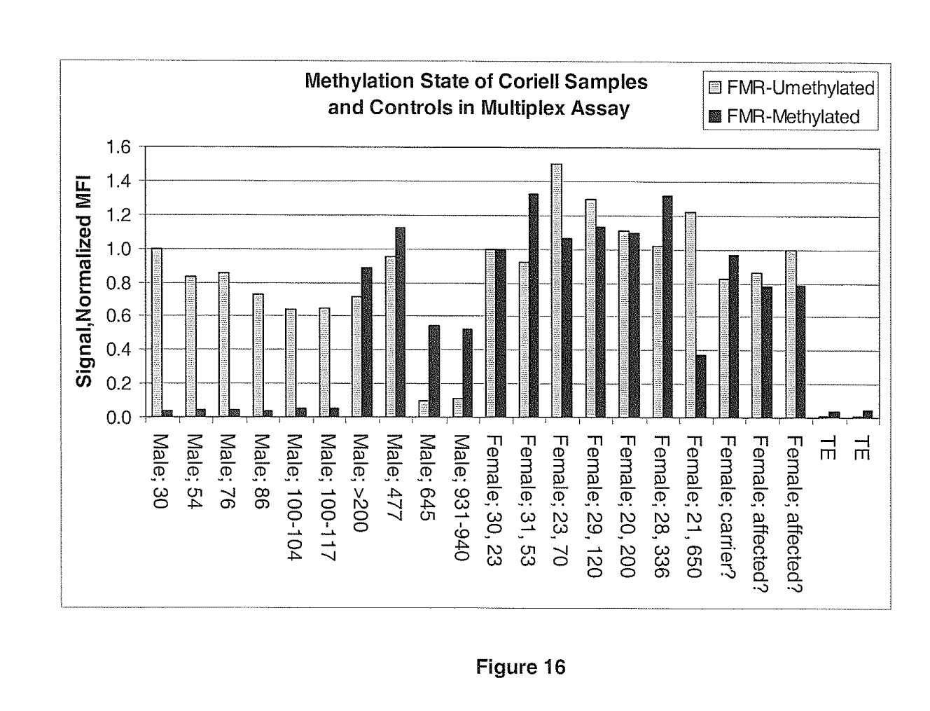

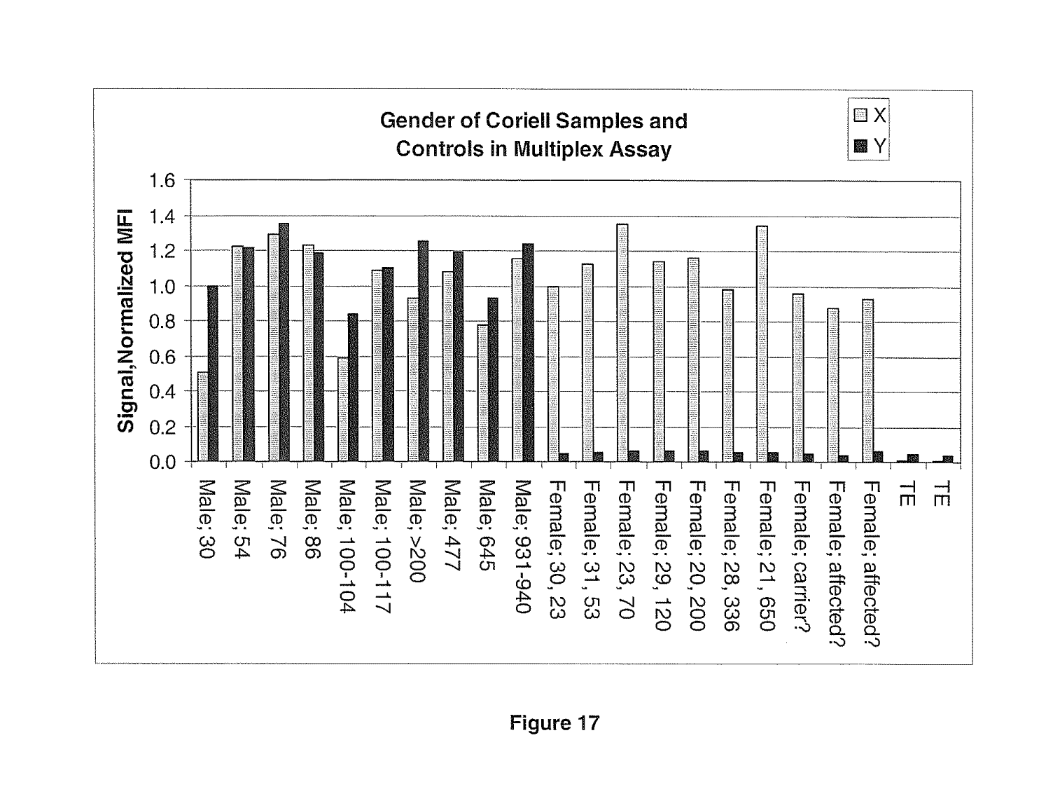

FIGS. 16 and 17 show graphical results from these experiments. For each graph, the vertical axis is the fluorescent signal for each of the samples as reported by the Luminex reading instrument as Median Fluorescent Intensity (MIFI) normalized to a normal male and a normal female. FIG. 16 shows the methylation signals from the four-plex bead hybridization. The signal from the encoded beads with capture probe for the unmethylated DNA from the FMR target locus are shown on the left side of the pair-wise signals for each sample, while the signals from the beads with the capture probe for methylated DNA from the target locus are shown on the right side of the pair-wise signals for each sample. FIG. 17 shows the chromosome specific amelogenin signals from the four-plex bead hybridization. The signal from the beads with the capture probe for the X chromosome amelogenin target locus are shown on the left side of the pair-wise signals for each sample, while the signals from the beads with the capture probe for Y chromosome amelogenin target locus are shown on the right side of the pair-wise signals for each sample.

Referring to FIG. 16, the methylation data shown is for the example multiplex assay run on samples. Male DNA samples were obtained from Fragile X affected males (Male>200; Male 477, Male 645; and Male 931-940, the numbers referring to the number of CGG repeats) and unaffected males (Male 30, Male 54, Male 76, Male 86; Male 100-104 and Male 100-117). Female DNA samples were obtained from two Fragile X affected females (with undefined CGG repeats), a Female carrier and unaffected females with various COG repeats (Female 30, 23; Female 31, 53; Female, 23, 70; Female 29, 120; Female 20, 200; Female 28, 336 and Female 21, 650). Hybridization control samples were TE buffer (10mM Tris-HCL (pH 8.0), 1 mM EDTA). As can be seen, the methylation-positive signal was increased in affected males, in comparison with unaffected males, while the methylation-negative signal was decreased in affected males, in comparison to unaffected males. The methylation signal in affected females was not discernibly different in comparison with unaffected females.

Referring to FIG. 17, the chromosome specific amelogenin signal for each sample can be used to determine the gender of each sample. Male samples have amelogenin signals for both AMEX and AMEY. Whereas the female samples only have signals for AMEX.

### Example 4

This example describes use of Prader-Willi/Angelman syndromes-associated methylation imprinted CpG islands as a representative target genomic locus. Prader-Willi syndrome is caused by the loss of paternally expressed genes within chromosome 15q11-q13.4. The majority of cases are attributed to a deletion of 15q11-q13 on the paternally inherited chromosome (over 70%), and the remaining cases are attributed to maternal uniparental disomy (over 20%) and imprinting center defects (<1%). Conversely, the Angelman syndrome is caused by the loss of maternally express genes within chromosome 15q11-q13.4. The majority of the Angelman cases are attributed to a deletion of 15q11-q13 on the maternally inherited chromosome (over 70%), and the remaining cases are attributed to paternal uniparental disomy (over 5%) and imprinting center defects (<1%).

Uniparental disomy refers to the situation in which two copies of a chromosome come from the same parent, instead of one copy coming from each the mother and father. Detecting the methylation state within the 15q11.2-13 locus can be used to determine the presence of maternal (methylated) and/or paternal (unmethylated) alleles. Using the methods described herein, CpG dinucleotide sequences are analyzed for methylation state. The relative amount of methylation, and corresponding presence of maternal or paternal alleles, can be used to determine the aberrant methylation state of samples from patients affected with the Prader-Willi or Angelman syndrome.

This example describes an assay performed on the SNRPN promoter locus as a representative methylation target genomic locus, using samples collected from patients affected with the Prader-Willi or Angelman syndrome.

The experimental work was performed essentially as is described in Example 2, with variations as described below.

The SNRPN specific PCR conditions were essentially as described in Example 1 with the following exceptions. The forward and reverse primers are specific to the genomic locus but non-specific to the pre-conversion methylation state of the sample DNA. The forward primer has no CpG dinucleotides and therefore will bind to all converted DNA template. Each of the two reverse primers was designed for converted methylated or unmethylated DNA respectively. The primer sequences were:

(SEQ ID No. 12)

| | |
|---|---|
| Forward: | TGT TTA TGG TTT TTA GAG GTT T |

(SEQ ID No. 13)

| | |
|---|---|
| Reverse 1: | CCA CCA ACA CAA CTA ACC TTA C |

(SEQ ID No. 14)

| | |
|---|---|
| Reverse 2: | CCA CCG ACA CAA CTA ACC TTA C |

In this example, the PCR cycles for the SNRPN target were:
95° C. for 15 minutes to activate the polymerase
Then the following temperature sequence:
94° C. melting, 30sec
55° C. annealing, 1 min
72° C. extension, 1 min
for 40 cycles to produce amplified converted DNA.

The capture probe sequence for the paternal unmethylated SNRPN specific bisulphite converted amplified DNA was: TAT GTT TGT GTG GTT GTA GAG GTA GGT TGG TGT GTA T (SEQ ID No. 15)

The capture probe sequence for the maternal methylated SNRPN specific bisulphite converted amplified DNA was: CCG ACG ACC GCT CCA CTC TAC GCC AAA CTC GCT ACA ACA ACG AC (SEQ ID No. 16)

The hybridization and detection of the SNRPN amplified converted DNA using encoded bead sets with either the immobilized maternal specific capture probe or the paternal specific capture was done as described in Example 2.

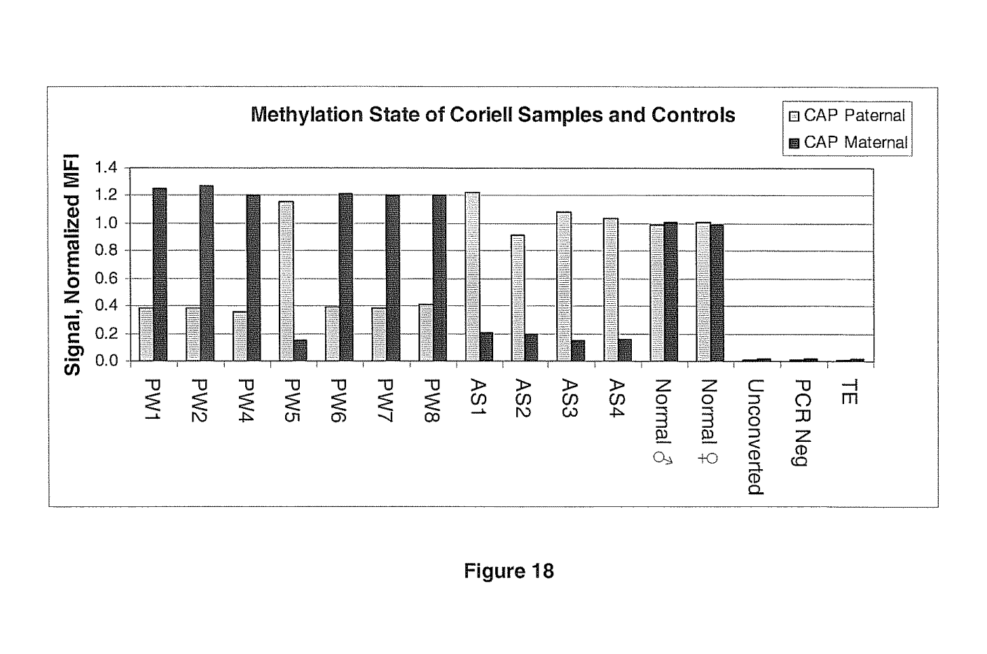

FIG. 18 show graphical results from these experiments. The vertical axis is the fluorescent signal for each of the samples as reported by the Luminex reading instrument as Median Fluorescent Intensity (MIFI) normalized to a normal male and a normal female. The signal from the encoded beads with capture probe for the unmethylated DNA from the SNRPN target locus are shown on the left side of the pair-wise signals for each sample and represents detection of the paternal allele. The signals from the beads with the capture probe for methylated DNA from the target locus are shown on the right side of the pair-wise signals for each sample and represents detection of the maternal allele.

Referring to FIG. 18, the normal male (♂) and the normal female (♀) samples have both an unmethylated and a methylated allele for SNRPN indicating respectively, the presence of both the paternal and maternal chromosome. Coriell DNA samples from patients affected with Prader-Willi (PW1, PW2, PW4, PW6, PW7 and PW8) have a prominent methylated signal and much reduced unmethylated signal indicating a missing paternal allele. Whereas, DNA samples from patients affected with Angelman (AS1, AS2, AS3, and AS4) have a prominent unmethylated signal and much reduced methylated signal indicating the absence of the maternal allele. One unaffected (PW5), father of PW6 and PW7, has a methylation pattern resembling affected Angelman patients. The molecular documentation from this patient indicates a maternal deletion of SNRPN, which was passed paternally to PW6 and PW7. PCR controls that do not produce PCR product (unconverted DNA or PCR negative water blank) and the hybridization negative control (TE buffer) have little signal.

Any patents or publications mentioned in this specification are incorporated herein by reference to the same extent as if each individual publication is specifically and individually indicated to be incorporated by reference.

The compositions, methods and kits described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for detecting an aggregate cytosine methylation state in a target genomic locus, comprising:
treating a DNA sample comprising the target genomic locus with bisulfite to convert unmethylated cytosine to uracil, producing converted DNA;
amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine;
labeling the amplified converted DNA with a label;
contacting the labeled amplified converted DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate;
detecting the label; and
detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.

2. The method of claim 1, wherein the encoded substrates are encoded particles.

3. The method of claim 1, wherein the encoded substrates are elements of a microarray.

4. The method of claim 1, wherein the amplifying is by polymerase chain reaction.

5. The method of claim 1, wherein the primers that flank the target genomic locus comprise a composite primer.

6. The method of claim 1, wherein the primers that flank the target genomic locus comprise a non-methylation specific primer.

7. The method of claim 1, wherein the primers that flank the target genomic locus comprise a methylation-specific primer.

8. The method of claim 1, wherein the labeling is performed during the amplifying.

9. The method of claim 8, wherein the labeling comprises amplifying converted DNA using a labeled primer.

10. The method of claim 8, wherein the labeling comprises amplifying converted DNA using labeled nucleotides.

11. The method of claim 1, wherein the labeling is performed after the amplifying.

12. The method of claim 11, wherein the labeling comprises binding of a labeled probe specific for the amplified converted DNA.

13. The method of claim 1, wherein the target genomic locus is associated with a disorder selected from the group consisting of: Fragile X mental retardation syndrome, Prader-Willi syndrome, Angelman sydrome, Beckwith-Wiedemann syndrome, and Russell-Silverman syndrome.

14. The method of claim 1, wherein the target genomic locus is associated with a disorder selected from the group consisting of: diabetes, cancer, multiple sclerosis and schizophrenia.

15. The method of claim 1, wherein the first and second capture probes are oligonucleotides.

16. The method of claim 1, further comprising:
treating a reference DNA sample comprising the target genomic locus with bisulfite to convert unmethylated cytosine to uracil, producing converted reference DNA;
amplifying converted reference DNA using primers that flank the target genomic locus to produce amplified converted reference DNA, wherein an unprotected amplification product is produced when the target genomic locus sample lacks methylated cytosine, and a protected amplification product is produced when the target genomic locus contains methylated cytosine;
labeling the amplified converted reference DNA with a label;
contacting the labeled amplified converted reference DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate;
detecting the label;
detecting the encoding of the first and second encoded substrates, wherein association of the detected label with the first encoded substrate indicates that the target genomic locus in the reference DNA sample has a negative aggregate cytosine methylation state and wherein association of the detected label with the second encoded substrate indicates that the target genomic locus in the reference DNA sample has a positive aggregate cytosine methylation state; and
comparing the aggregate cyosine methylation states of the DNA sample and the reference DNA sample.

17. The method of claim 1, further comprising:
amplifying converted DNA using primers that flank a second target genomic locus to produce amplified converted DNA, wherein an unprotected amplification product is produced when the second target genomic locus in the DNA sample lacks methylated cytosine, and a protected amplification product is produced when the second target genomic locus in the DNA sample contains methylated cytosine;
labeling the amplified converted DNA with a label;
contacting the labeled amplified converted DNA with (I) a third capture probe, wherein the third capture probe is selective for the unprotected amplification product of the second target genomic locus and is associated with a third encoded substrate, and (ii) a fourth capture probe, wherein the fourth capture probe is selective for the protected amplification product of the second target genomic locus and is associated with a fourth encoded substrate;
detecting the label; and
detecting the encoding of the third and fourth encoded substrates, wherein association of the detected label with the third encoded substrate indicates that the second target genomic locus in the DNA sample has a negative aggregate cytosine methylation state and wherein association of the detected label with the fourth encoded substrate indicates that the second target genomic locus in the DNA sample has a positive aggregate cytosine methylation state.

18. The method of claim 1, further comprising:
amplifying converted DNA using primers that flank a gender-specific target genomic locus to produce a gender-specific amplified converted DNA;
contacting the gender-specific amplified converted DNA with (i) a first capture probe selective for the female-specific amplification product, the first capture probe attached to a first gender-specific encoded substrate, and (ii) a second capture probe selective for the male-specific amplification product, the second capture probe attached to a second gender-specific encoded substrate;
labeling the gender-specific amplified converted DNA with a label;
detecting the label; and
detecting the encoding of the encoded substrates to produce a gender result, wherein association of the detected label with the first gender-specific encoded substrate indicates that the DNA sample was obtained from a female subject and wherein association of the detected label with the second gender-specific encoded substrate indicates that the DNA sample was obtained from a male subject.

19. The method of claim 18, wherein the gender-specific target genomic locus is an amelogenin genomic locus.

20. A kit for detecting the aggregate methylation state of a target genomic locus; comprising:
an encoded particle set comprising attached capture probes specific for a target nucleic acid comprising more than one CpG island; and
instructional material for use of the encoded particle set in a method according to claim 1.

21. A composition, comprising:
a complex of a capture probe attached to an encoded substrate and labeled unprotected amplification product, wherein the complex indicates that the unprotected amplification product is derived from a target genomic locus in a DNA sample having a negative aggregate cysteine methylation state.

22. A composition, comprising:
a complex of a capture probe attached to an encoded substrate and labeled protected amplification product, wherein the complex indicates that the protected amplification product is derived from a target genomic locus in a DNA sample having a positive aggregate cysteine methylation state.

23. A method for screening a subject for a disease or disorder characterized by methylation of a genomic locus, comprising:
treating a DNA sample obtained from the subject comprising the target genomic locus with bisulfite to convert unmethylated cytosine to uracil, producing converted DNA;
amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine;
labeling the amplified converted DNA with a label;
contacting the labeled amplified converted DNA with (i) a first capture probe, wherein the first capture probe is selective for the unprotected amplification product and is associated with a first encoded substrate, and (ii) a second capture probe, wherein the second capture probe is selective for the protected amplification product and is associated with a second encoded substrate;
detecting the label; and
detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.

24. A method for detecting a cytosine methylation state in a target genomic locus, comprising:
treating a DNA sample comprising the target genomic locus with bisulfite to convert unmethylated cytosine to uracil, producing converted DNA;
amplifying converted DNA using primers that flank the target genomic locus to produce amplified converted DNA, wherein an unprotected amplification product is produced when the target genomic locus lacks methylated cytosine, and a protected amplification product is produced when the target genomic locus contains methylated cytosine;
labeling the amplified converted DNA with a label; contacting the labeled amplified converted DNA with (i) a first capture probe, wherein the first capture probe is selective for the unprotected amplification product to produce a complex of the first capture probe and the unprotected amplification product and (ii) a second capture probe, wherein the second capture probe is selective for the protected amplification product to produce a complex of the second capture probe and the protected amplification product;
associating the complex of the first capture probe and the unprotected amplification product and with a first encoded substrate;
associating the complex of the second capture probe and the protected amplification product and with a second encoded substrate; detecting the label; and
detecting the encoding of the first and second encoded substrates, wherein association of the detected label with the first encoded substrate indicates that the target genomic locus in the DNA sample has a negative aggregate cytosine methylation state and wherein association of the detected label with the second encoded substrate indicates that the target genomic locus in the DNA sample has a positive aggregate cytosine methylation state.
